(19) United States
(12) Patent Application Publication
(10) Pub. No.: US 2024/0261330 A1
(43) Pub. Date: Aug. 8, 2024

(54) CD19-Directed Chimeric Antigen Receptor Constructs

(71) Applicant: Sorrento Therapeutics, Inc., San Diego, CA (US)

(72) Inventors: Qiangzhong Ma, San Diego, CA (US); Wenzhong Guo, San Diego, CA (US); Bei Bei Ding, San Diego, CA (US); Yanliang Zhang, San Diego, CA (US)

(73) Assignee: Sorrento Therapeutics, Inc., San Diego, CA (US)

(21) Appl. No.: 18/248,079

(22) PCT Filed: Oct. 11, 2021
(86) PCT No.: PCT/US2021/054437

§ 371 Date: Apr. 6, 2023

Related U.S. Application Data
(60) Provisional application No. 63/090,440, filed on Oct. 12, 2020.

(51) Int. Cl. A61K 35/17; A61K 39/00; A61P 35/02; C07K 16/28; C12N 15/86

(52) U.S. Cl. CPC A61K 35/17; A61K 39/4611; A61K 39/4631; A61K 39/464412; A61P 35/02; C07K 16/2803; C12N 15/86; A61K 2239/13; A61K 2239/17; A61K 2239/21; A61K 2239/22; A61K 2239/48; C07K 2317/56; C07K 2317/622; C07K 2319/02; C07K 2319/03; C07K 2319/41; C12N 2740/10043

(57) ABSTRACT

Disclosed in the present application are CAR constructs that encode domains of anti-CD19 antibodies, T cells that include and express the CD19 CAR constructs, and methods of use, such as methods of treating disease, including hematological cancers, using CAR-T cells that express the CD19 CAR constructs.

This application is a national phase entry pursuant to 35 U.S.C. § 371 of International Application No. PCT/US2021/054437, filed Oct. 11, 2021, which claims benefit of priority under 35 U.S.C. § 119 to U.S. provisional application No. 63/090,440, filed Oct. 12, 2020, entitled “CD19-Directed Chimeric Antigen Receptor Constructs”, the contents of each of which are incorporated by reference herein in their entireties.

### SEQUENCE LISTING

The instant application contains a Sequence Listing named 2021-10-11_01223-0087-00PCT_Sequence_Listing_ST25.txt which was created on Oct. 11, 2021 and is 56,997 bytes in size. The Sequence Listing has been filed electronically in ASCII format and is hereby incorporated by reference in its entirety.

### TECHNICAL FIELD

The present disclosure provides a CAR (chimeric antigen receptor) construct directed to CD19 and T cells populations that express the CD19 CAR. The T cells demonstrate cytotoxicity toward tumor cells that express CD19 and can be used in the treatment of disease, such as cancer.

### BACKGROUND

Chimeric antigen receptors (CARs) are synthetic receptors in which a targeting moiety is associated with one or more signaling domains in a single fusion molecule. T-cells having novel specificities have been generated through the genetic transfer of constructs encoding CARs that can specifically bind a target of interest. CARs have successfully been used to engineer T cells to be directed against antigens expressed at the surface of tumor cells from various malignancies including lymphomas and solid tumors.

In general, the extracellular targeting moiety of a CAR consists of an antigen-binding domain of a single-chain antibody (scFv) comprising the light and heavy variable fragments of a monoclonal antibody joined by a flexible linker. The intracellular signaling domain for first generation CARs is derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signaling domains from co-stimulatory molecules including CD28, OX-40 (CD134), and 4-1BB (CD137) have been added either singly (second generation CARs) or in combination (third generation CARs) to enhance survival and increase proliferation of CAR-modified T cells.

T cells have the power to dispose of normal or malignant cells as seen in viral and autoimmune diseases and as also seen in the rare spontaneous remissions of cancer. However, T cells are easily tolerized to self or tumor antigens, and “immune surveillance” has failed to prevent tumor progression of every cancer that is clinically apparent. It is the goal of CAR-T studies to supply the specificities and affinities to a patient's T cells, without regard for their “endogenous” T cell receptor (TCR) repertoire, by providing an antibody-defined, anti-malignant cell marker recognition to kill malignant cells based on their expression of antigens recognized by the CAR.

Adoptive immunotherapy by infusion of T cells engineered CARs for redirected tumoricidal activity has been explored for the treating of metastatic cancer. CARs are constructed by joining the antigen recognition domains of an antibody with the signaling domains of receptors from T cells. Modification of T cells with CAR genes equips T cells with retargeted antibody-type antitumor cytotoxicity. Because killing is Major Histocompatibility Complex (MHC)-unrestricted, the approach offers a general therapy for all patients bearing the same antigen. T cells engineered with antigen specific CARs are called “CAR-T cells” or “T-bodies” (Eshar, et al., 1993 Proc. Nat'l. Acad. of Sci. USA 90(2):720-724). A first-generation CAR, immunoglobulin-T cell receptor (IgTCR), was engineered to contain a signaling domain (TCR-CD3ζ) that delivers an activation stimulus (signal 1) only (Gross, et al., 1989 Proc. Nat'l. Acad. of Sci. USA 86(24):10024-10028; Eshar, et al., 1993 Proc. Nat'l. Acad. of Sci. USA 90(2): 720-724; Haynes, et al., 2001 J Immunol. 166(1): 182-187). T cells engineered to express the first-generation CARs alone exhibit limited anti-tumor efficacy due to suboptimal activation. A 2nd generation CAR, immunoglobulinCD28-CD3ζ-T cell receptor (IgCD28TCR), incorporated a costimulatory CD28 (signal 2) into the first-generation receptor that increased the anti-tumor capacity of the CAR-T cells (Finney, et al., 1998 J Immunol. 161(6):2791-2797; Hombach, et al., 2001 J Immunol. 167(11):6123-6131; Maher, et al., 2002 Nat Biotechnol. 20(1):70-7; Emtage, et al., 2008 Clin Cancer Res. 14(24):8112-812; Lo, et al., 2010 Clin Cancer Res. 16(10):2769-2780).

The current protocol for treatment of patients using adoptive immunotherapy is based on autologous cell transfer. In this approach, T lymphocytes are recovered from patients, genetically modified or selected ex vivo, cultivated in vitro in order to amplify the number of cells if necessary and finally infused into the patient. In addition to lymphocyte infusion, the host (patient) may be manipulated in other ways that support the engraftment of the T cells or their participation in an immune response, for example pre-conditioning (with radiation or chemotherapy) and administration of lymphocyte growth factors (such as IL-2). Each patient receives an individually fabricated treatment, using the patient's own lymphocytes (i.e. an autologous therapy).

Alternatively, an allogeneic strategy can be used where T cells from a donor is infused into the patient. Allogeneic T cells from healthy donors can be transfected with a CAR construct in advance for treatment of cancer and avoid expensive and time-consuming preparation and testing of each individual patient's T cells. To avoid incompatibility between donor cells and the patient, the donor may be HLA matched with the patient or the allogeneic T cells expressing the CAR construct may be further genetically altered to eliminate expression of the endogenous T cell receptor.

CD19 is a 95 kD type II transmembrane glycoprotein of the immunoglobulin superfamily with a long C-terminal cytoplasmic domain that is expressed by cells of the B cell lineage. During ontogeny, CD19 appears on B-lineage committed stem cells and its expression increases with B cell maturation, with mature B cells expressing approximately three-fold the surface density of immature B cells. CD19 is also expressed on malignant B cells, and has been used as a target in anti-neoplastic therapies that originate from the B cell lineage, including for example Non-Hodgkin Lymphoma (NHL), acute lymphoblastic leukemia (ALL), and chronic lymphocytic leukemia (CLL).

### SUMMARY

The present disclosure provides a nucleic acid molecule encoding a chimeric antigen receptor (CAR) that comprises an anti-CD19 antibody capable of targeting tumor cells that express CD19. More specifically, the present disclosure provides a CD19 CAR nucleic acid construct in which the encoded CAR comprises a single chain antibody (scFv) that specifically binds CD19, in which the scFv antibody has a light chain variable (VL) domain having an amino acid sequence that is at least 95% homologous to the amino acid sequence of SEQ ID NO: 1 and a heavy chain variable (VH) domain having an amino acid sequence that is at least 95% homologous to the amino acid sequence of SEQ ID NO:2. The encoded CD19 CAR further includes a transmembrane domain and at least one intracellular signaling domain and preferably further includes a hinge region between the antigen binding protein and the transmembrane domain. In various embodiments the hinge region is a long hinge region, for example a hinge region of greater than about 65 amino acids in length (e.g., at least 70, at least 75, at least 80, or at least 85 amino acids in length), and may be derived from the hinge regions of two or more polypeptides. In some embodiments the hinge region comprises an amino acid sequence having at least 95%, at least 96%, at least 97%, at least 98%, or at least 99% identity to the amino acid sequence of SEQ ID NO:13.

A CD19 CAR encoded by a nucleic acid molecule as provided herein thus includes a single chain antibody derived from an antibody that specifically binds to CD19, in which the light chain variable (VL) domain comprises an amino acid sequence that is at least 95%, at least 96%, at least 97%, at least 98%, or at least 99% identical to the amino acid sequence of SEQ ID NO:1 and the heavy chain variable (VH) domain comprises an amino acid sequence that is at least 95%, at least 96%, at least 97%, at least 98%, or at least 99% identical to the amino acid sequence of SEQ ID NO:2; and further includes a transmembrane domain and at least one intracellular domain. The anti-CD19 single chain antibody of the CAR can in some embodiments comprise the amino acid sequence of SEQ ID NO: 12 or can comprise an amino acid sequence having at least 95%, at least 96%, at least 97%, at least 98%, or at least 99% identity to the amino acid sequence of SEQ ID NO:12. In alternative embodiments the anti-CD19 single chain antibody of the CAR can comprise the amino acid sequence of SEQ ID NO:22 or can comprise an amino acid sequence having at least 95%, at least 96%, at least 97%, at least 98%, or at least 99% identity to the amino acid sequence of SEQ ID NO:22.

A CD19 CAR as encoded by a nucleic acid construct provided herein includes at least one intracellular signaling domain, and preferably includes two intracellular signaling domains. In embodiments where the CD19 CAR includes a first and a second intracellular signaling domain the first signaling domain can be any of a CD28 intracellular signaling domain, an OX-40 (CD134) signaling domain, and a 4-1BB (CD137) signaling domain. The second signaling domain can be, for example, a CD3-ζ signaling domain. In some preferred embodiments, a first signaling domain is a CD28 signaling domain having the amino acid sequence of SEQ ID NO:7 or having an amino acid sequence with at least 95% identity to SEQ ID NO:7. In some preferred embodiments, a second signaling domain is a CD3-ζ signaling domain (SEQ ID NO:8) or a signaling domain derived therefrom having an amino acid sequence at least 95% identical to SEQ ID NO:8.

The CD19 CAR can include a transmembrane domain derived from a transmembrane polypeptide such as CD4, CD8, CD16, CD28, CD80, CD86, CD134, CD137, IgG1, or lgG4, as nonlimiting examples. In some embodiments a CD19 CAR encoded by a nucleic acid molecule as provided herein has a transmembrane domain derived from CD28, and can be a transmembrane domain having at least 95% identity to SEQ ID NO:6. In various examples, a CD19 CAR encoded by a nucleic acid construct as provided herein includes a CD8 hinge region positioned between the scFv and the transmembrane domain, for example a CD8α hinge region, and/or a CD28 hinge region. The hinge region can include, for example, an amino acid sequence having at least 95% identity to SEQ ID NO:4 and an amino acid sequence having at least 95% identity to SEQ ID NO:5, for example a hinge region have sequences derived from both CD8 and CD28, e.g., where the hinge region includes an amino acid sequence having at least 95% identity to SEQ ID NO: 13.

Preferably, the nucleic acid construct further includes a nucleic acid sequence encoding a signal peptide at the N-terminus of the CAR that may be removed when the synthesized CAR is localized to the cell membrane. For example, the signal peptide of SEQ ID NO:9 may be used, or a signal peptide having at least 95% identity thereto, or another signal peptide may be used.

In some embodiments, the construct can optionally include a sequence encoding a peptide tag for detection of the synthesized CAR, for example, a myc tag (SEQ ID NO: 10) or another peptide tag. In some embodiments a peptide tag is positioned at the N-terminus of a mature CD19 CAR, for example between the signal peptide and scFv of a precursor CAR encoded by a construct as provided herein.

The CD19 CAR encoded by a nucleic acid molecule as provided herein can in some embodiments be the CAR of SEQ ID NO: 14 or a CAR having at least 95%, at least 96%, at least 97%, at least 98%, or at least 99% identity to the CAR of SEQ ID NO: 14. For example, the nucleic acid molecule can include the nucleic acid sequence of SEQ ID NO: 15 or a nucleic acid sequence having at least 65%, 70%, 75%, 80%, 85%, 90%, or 95% identitiy thereto, where the nucleic acid sequence encodes a CD19 CAR having at least 95% identity to SEQ ID NO: 14. In alternative embodiments a CD19 CAR encoded by a nucleic acid molecule provided herein can be the CAR of SEQ ID NO:24 or a CAR having at least 95%, at least 96%, at least 97%, at least 98%, or at least 99% identity to the CAR of SEQ ID NO:24. For example, the nucleic acid molecule can include the nucleic acid sequence of SEQ ID NO:25 or a nucleic acid sequence having at least 65%, 70%, 75%, 80%, 85%, 90%, or 95% identitiy thereto, where the nucleic acid sequence encodes a CD19 CAR having at least 95% identity to SEQ ID NO:24.

A nucleic acid molecule comprising a sequence encoding a CD19 CAR as disclosed herein can be, as nonlimiting examples, a DNA molecule or RNA molecule, and can optionally include one or more modified nucleotides and/or one or more backbone modifications. The nucleic acid molecule can be a linear nucleic acid molecule or can be a circular nucleic acid molecule, and can be double-stranded or single-stranded. A nucleic acid molecule as provided herein can be a vector that includes the CAR-encoding sequence. The nucleic acid molecule can be a viral vector or a viral genome (in circular or linear form). In some embodiments the vector may be a lentiviral or retroviral transfer vector, for example, a replication-defective lentiviral or retroviral (e.g., gammaretroviral) transfer vector, that includes a lentiviral or retroviral packaging sequence. Further included herein is a lentivirus or retrovirus that includes a CD19 CAR encoding nucleic acid sequence. In some embodiments the retrovirus is a gammaretrovirus. The CAR-encoding sequence of a nucleic acid molecule as provided herein can be operably linked to expression control sequences, such as but not limited to a promoter. A promoter operably linked to a CD19 CAR-encoding sequence is preferably a promoter active in a human cell and in some embodiments can be a retroviral promoter, e.g., an LTR promoter of a retrovirus.

In various embodiments provided herein is a gammaretroviral vector comprising a nucleic acid sequence encoding a CD19 CAR, such as the CD19 CAR of SEQ ID NO: 14 or a CAR having at least 95%, at least 96%, at least 97%, at least 98%, or at least 99% identity to the CAR of SEQ ID NO:14. In some exemplary embodiments the gammaretroviral vector includes a sequence that encodes the CD19 CAR of SEQ ID NO: 14 and has the sequence of SEQ ID NO:16, or has a sequence having at least 96%, at least 97%, at least 98%, or at least 99% identity to SEQ ID NO:16.

In further embodiments provided herein is a gammaretroviral vector comprising a nucleic acid sequence encoding a CD19 CAR, such as the CD19 CAR of SEQ ID NO:24 or a sequence having at least 95%, at least 96%, at least 97%, at least 98%, or at least 99% identity identity to the CAR of SEQ ID NO:24. In some exemplary embodiments the gammaretroviral vector encodes the CD19 CAR of SEQ ID NO:24 and has the sequence of SEQ ID NO:26, or has a sequence having at least 96%, at least 97%, at least 98%, or at least 99% identity to SEQ ID NO:26.

In a further aspect, provided herein is a CD19 chimeric antigen receptor (CAR) that comprises: an anti-CD19 scFv in which the scFv antibody has a V_{L} domain having an amino acid sequence that is at least 95% homologous to the amino acid sequence of SEQ ID NO: 1 and a V_{H} domain having an amino acid sequence that is at least 95% homologous to the amino acid sequence of SEQ ID NO:2. The CD19 CAR further includes a transmembrane domain and at least one intracellular signaling domain and further includes a hinge region between the antigen binding protein and the transmembrane domain, for example a long hinge region that comprises an amino acid sequence having at least 95%, at least 96%, at least 97%, at least 98%, or at least 99% identity to the amino acid sequence of SEQ ID NO:13. The transmembrane domain can be, as nonlimiting examples, a transmembrane domain of a CD4, CD8, CD16, CD28, CD80, CD86, CD134, CD137, IgG1, or lgG4 polypeptide, or a transmembrane domain having at least 95% identity to any thereof. The CAR preferably includes two intracellular signaling domains, which can be, for example, a CD28 signaling domain and a CD3ζ signaling domain. In some embodiments the CD19 CAR comprises an amino acid sequence having at least 95%, at least 96%, at least 97%, at least 98%, or at least 99% identity to the amino acid sequence of SEQ ID NO:27. In some embodiments the CD19 CAR comprises an amino acid sequence having at least 95%, at least 96%, at least 97%, at least 98%, or at least 99% identity to the amino acid sequence of SEQ ID NO:23. The CD19 CAR can be an isolated polypeptide. The CD19 CAR can be a polypeptide inserted into the membrane of a transgenic cell, such as an engineered host cell as disclosed herein.

The present disclosure further provides a host cell, or a population of host cells, that includes a CD19 CAR construct, such as any provided herein. In various embodiments the CD19 CAR can comprise: (i) an scFv that binds to CD19, wherein the scFv comprises a light chain variable (VL) domain comprising an amino acid sequence that is at least 95% identical to the amino acid sequence of SEQ ID NO: 1 and a heavy chain variable (VH) domain comprising an amino acid sequence that is at least 95% identical to the amino acid sequence of SEQ ID NO:2; (ii) a transmembrane domain; and (iii) at least one intracellular signaling domain. In various embodiments the host cell(s) comprise a CD19 CAR construct as disclosed herein that includes (i) an scFv that binds to CD19, wherein the scFv comprises a light chain variable (VL) domain comprising an amino acid sequence that is at least 95% identical to the amino acid sequence of SEQ ID NO: 1 and a heavy chain variable (VH) domain comprising an amino acid sequence that is at least 95% identical to the amino acid sequence of SEQ ID NO:2; (ii) a transmembrane domain; and (iii) two intracellular signaling domains, where a first intracellular signaling domain is a CD28, OX-40, or 4-1BB intracellular signaling domain and the second intracellular signaling domain is a CD3-ζ signaling domain. In various embodiments the CAR further includes a hinge region between the anti-CD19 scFv and the transmembrane domain that in some embodiments can be a long hinge region of greater than about 65 amino acids in length and may comprise a sequence derived from a CD8 hinge sequence and a sequence derived from a CD28 hinge sequence. For example, the host cell or population of host cells can include a non-native nucleic acid sequence encoding a CD19 CAR that includes an anti-CD19 scFv having at least 95% identity to SEQ ID NO:12 or at least 95% identity to SEQ ID NO:22 and can further include a hinge region having at least 95% identity to SEQ ID NO:13; a transmembrane domain having at least 95% identity to SEQ ID NO:6; a first signaling domain having at least 95% identity to SEQ ID NO:7; and a second signaling domain having an amino acid sequence at least 95% identical to SEQ ID NO:8. Preferably, the CAR construct further includes a signal peptide-encoding sequence at the N-terminus of the CAR-encoding sequence, e.g., a sequence encoding SEQ ID NO:9 or a signal peptide having at least 95% identity thereto. The construct can optionally also include a sequence encoding a peptide tag for detection of the synthesized CAR, such as, for example, a myc tag (SEQ ID NO:10) or another peptide tag.

A cell engineered to express a nucleic acid molecule that includes a sequence encoding a CD19 CAR as provided herein can specifically bind CD19. Exemplary nucleic acid sequences encoding CD19 CARs include SEQ ID NO:25 and SEQ ID NO:15.

The host cell or population of host cells can be, for example, T cells, and can be pan T cells which may be PBMC-derived T cells, placenta-derived T cells, or cord blood derived T cells, or the T cells may be a subpopulation of T cells isolated from PBMCs, placenta, or cord blood. In various embodiments, the host cells are cells transduced with a lentvirus or retrovirus (e.g., a gammaretrovirus) having a nucleic acid sequence that encodes a CD19 CAR as provided herein. The nucleic acid sequence encoding the CD19 chimeric antigen receptor (CAR) is operably linked to a promoter which directs expression of the CD19 CAR construct in the transduced host cell or population of host cells. The promoter in some embodiments can be a retroviral promoter, for example, a promoter of the LTR of the retroviral vector.

In some examples a host cell or population of host cells includes a nucleic acid sequence encoding SEQ ID NO:14 or an amino acid sequence having at least 95%, at least 96%, at least 97%, at least 98%, or at least 99% identity to SEQ ID NO:14. In some examples a host cell or population of host cells includes the nucleic acid sequence of SEQ ID NO: 15 or an amino acid sequence having at least 85%, at least 90%, at least 95%, at least 96%, at least 97%, at least 98%, or at least 99% identity to SEQ ID NO:15.

In some examples a host cell or population of host cells includes a nucleic acid sequence encoding SEQ ID NO:24 or an amino acid sequence having at least 95%, at least 96%, at least 97%, at least 98%, or at least 99% identity to SEQ ID NO:24. In some examples a host cell or population of host cells includes the nucleic acid sequence of SEQ ID NO:25 or an amino acid sequence having at least 85%, at least 90%, at least 95%, at least 96%, at least 97%, at least 98%, or at least 99% identity to SEQ ID NO:25.

The CAR-encoding nucleic acid sequence may be inserted into the genome of the cell. In some examples, the host cells, for example T cells, are transduced via a retrovirus engineered to include a gene encoding a CD19 CAR as disclosed herein, preferably operably linked to a promoter, which may be a retroviral promoter (e.g., a promoter of a retroviral LTR) and the transduced host cells include a retrovirally-mediated insertion of the nucleic acid sequence encoding the CD19 CAR. The transduced host cells may also include additional retroviral sequences associated with the CAR construct, such as for example, at least a portion of a retroviral (e.g., gammaretroviral) packaging signal.

The host cell or a population of host cells provided herein express the CD19 chimeric antigen receptor (CAR) construct, where the expressed CD19 CAR on the host cell or population of host cells specifically binds CD19. The T cell or a population of T cells that express the CD19 chimeric antigen receptor (CAR) construct, preferentially kill (are cytotoxic toward) cells (e.g., target cells) that express CD19.

The present disclosure provides a host cell, or a population of host cells, which express a CD19 CAR that includes a light chain variable (VL) domain comprising an amino acid sequence that is at least 95% identical to the amino acid sequence of SEQ ID NO: 1 and a heavy chain variable (VH) domain comprising an amino acid sequence that is at least 95% identical to the amino acid sequence of SEQ ID NO:2; a hinge region, a transmembrane domain; and at least one intracellular domain. The host cell or population of host cells can express a CD19 CAR such as any disclosed herein, for example, a CD19 CAR that includes a CD19 scFv having at least 95% identity to SEQ ID NO: 12 and further includes a hinge region having at least 95% identity to SEQ ID NO:4 and an amino acid sequence having at least 95% identity to SEQ ID NO:5; a transmembrane domain having at least 95% identity to SEQ ID NO:6; a first signaling domain having at least 95% identity to SEQ ID NO:7; and a second signaling domain having an amino acid sequence at least 95% identical to SEQ ID NO:8. The CAR can optionally also include a peptide tag for detection of the synthesized CAR, such as, for example, a myc tag (SEQ ID NO:10) or another peptide tag. The peptide tag in some embodiments may be N-terminal to the scFv of the CAR.

A host cell or population of host cells that includes a CD19 CAR construct as provided herein can have one or more genes that encodes a T cell receptor (TCR) subunit that is disrupted. For example, one or more of a TCRα or TCRβ gene may be mutated such that no functional subunit is produced. TCR subunit genes may be disrupted for example with CRISPR/Cas methods, Talens, or zinc finger nucleases.

In some embodiments, a host cell or population of host cells that express the CD19 CAR, can be a T cell (or a population thereof), which may be, for example, a peripheral blood derived T cell (or a population thereof), a placenta-derived T cell (or a population thereof) or a cord blood derived T cell (or a population thereof).

The present disclosure further provides a method for treating a cancer or inhibiting tumor growth in a subject in need of a treatment comprising: administering to the subject a population of T cells expressing a CD19 CAR as provided herein. The T cells administered to the subject are host cells as described herein that include a nucleic acid molecule that encodes a CD19 CAR as provided herein. The T cells can be, for example, peripheral blood-derived T cells, or may be T cells derived from placenta and/or cord blood.

The present disclosure therefore provides a method for treating a cancer or inhibiting tumor growth in a subject in need of a treatment comprising: administering to the subject a population of T cells expressing a CD19 CAR as disclosed herein, where the CAR includes a light chain variable (VL) domain comprising an amino acid sequence that is at least 95% identical to the amino acid sequence of SEQ ID NO: 1 and a heavy chain variable (VH) domain comprising an amino acid sequence that is at least 95% identical to the amino acid sequence of SEQ ID NO:2; a hinge region; a transmembrane domain; and an intracellular region comprising two intracellular domains. The host cell or population of host cells can include a nucleic acid sequence encoding a CD19 CAR such as any disclosed herein, and can express, for example, a CD19 CAR that includes an anti-CD19 scFv having at least 95% identity to SEQ ID NO: 12 and further includes: a hinge region having at least 95% identity to SEQ ID NO: 13; a transmembrane domain having at least 95% identity to SEQ ID NO:6; a first signaling domain having at least 95% identity to SEQ ID NO:7; and a second signaling domain having an amino acid sequence at least 95% identical to SEQ ID NO:8. The CAR expressed by the T cells can optionally include a peptide tag for detection of the expressed CAR, such as, for example, a myc tag (SEQ ID NO:10) or another peptide tag.

The present disclosure provides methods for conducting adoptive cell therapy by administering to a subject genetically engineered cells expressing any of the provided CD19 CARs. The methods provided herein include, for example, administering to a human subject a host cell which expresses a CD19 CAR described herein (or a host cell transduced to include a nucleic acid sequence encoding a CD19 CAR as described herein). The present disclosure provides methods for treating a cancer or inhibiting tumor growth in a subject in need of treatment comprising administering to the subject a population of T cells expressing a CD19 CAR such as any provided herein. In some embodiments the CD19 CAR expressed by the T cells comprises an amino acid sequence having at least 95% identity to the amino acid sequence of SEQ ID NO:24.

Preferably, the disorder is cancer, including, but not limited to hematologic breast cancer, ovarian cancer, prostate cancer, head and neck cancer, lung cancer, bladder cancer, melanoma, colorectal cancer, pancreatic cancer, lung cancer, liver cancer, renal cancer, esophageal cancer, leiomyoma, leiomyosarcoma, glioma, and glioblastoma.

In various embodiments the cancer is a hematologic cancer selected from the group consisting of acute lymphocytic leukemia (ALL), B chronic lymphocytic leukemia (B-CLL), non-Hodgkin's lymphoma (NHL), and Burkitt's lymphoma (BL), T cell lymphoma (TCL), acute myeloid leukemia (AML), hairy cell leukemia (HCL), Hodgkin's Lymphoma (HL), and chronic myeloid leukemia (CML). In some embodiments the hematologic cancer is selected from a group consisting of non-Hodgkin's lymphoma (NHL), Burkitt's lymphoma (BL), B chronic lymphocytic leukemia (B-CLL), and acute lymphocytic leukemia (ALL).

### DESCRIPTION OF THE DRAWINGS

FIG. 1A is a schematic representation of a construct encoding CD19 CARs created by linking an scFv of an anti-CD19 antibody to the hinge regions of CD8α and CD28, the CD28 transmembrane domain (TMD) and the CD28 and CD3ζ intracellular signaling domains. A signal peptide and a myc tag for detecting CAR expression were added to the N-terminus. FIG. 1B is a schematic representation of a construct encoding CD19 CARs created by linking an scFv of an anti-CD19 antibody to the hinge regions of CD8α and CD28, the CD28 transmembrane domain (TMD) and the CD28 and CD3ζ intracellular signaling domains. A signal peptide was added to the N-terminus. Figures are not to scale.

FIG. 2 provides a vector map of a retroviral vector that includes a nucleic acid sequence encoding a CAR as depicted in FIG. 1B.

FIG. 3 provides flow cytometric analysis of T cell populations transduced in a first experiment to include the CD19 CAR construct of SEQ ID NO: 15 (CD19 CAR2-63) stained with an antibody to CD3 to detect expression of the T cell receptor (Y axis) and with CD19 antigen linked to an Fc region to detect CD19 CAR expression (X axis). The top graph shows non-transduced control T cells, the middle graph shows T cells isolated from PBMCs transduced with CD19 CAR2-63 and the bottom graph shows T cells isolated from cord blood (CB).

FIG. 4 provides flow cytometric analysis of T cell populations transduced in a second experiment to include the CD19 CAR construct of SEQ ID NO: 15 (CD19 CAR2-63) recognized by CD19 antigen linked to an Fc region to detect CD19 CAR expression (Y axis). The upper left graph shows non-transduced control T cells isolated from PBMCs, and the upper right graph shows T cells isolated from PBMCs transduced with CD19 CAR2-63. The lower left graph shows non-transduced control T cells isolated from CB, and the upper right graph shows T cells isolated from CB transduced with CD19 CAR2-63.

FIG. 5 shows production of interferon gamma (IFNγ) by non-transduced T cells isolated from PBMCs and CB and T cells transduced with CD19 CAR2-63 that were isolated from PBMCs and CB. The T cells were cultured with K562 tumor cells that do not express CD19, with K562 tumor cells that were engineered to express CD19, and with Raji tumor cells that express CD19.

FIG. 6 shows production of interleukin 2 (IL-2) by non-transduced T cells isolated from PBMCs and CB and T cells transduced with CD19 CAR2-63 that were isolated from PBMCs and CB. The T cells were cultured with K562 tumor cells that do not express CD19, with K562 tumor cells that were engineered to express CD19, and with Raji tumor cells that express CD19.

FIG. 7 shows production of tumor necrosis factor alpha (TNFα) by non-transduced T cells isolated from PBMCs and CB and T cells transduced with CD19 CAR2-63 that were isolated from PBMCs and CB. The T cells were cultured with K562 tumor cells that do not express CD19, with K562 tumor cells that were engineered to express CD19, and with Raji tumor cells that express CD19.

FIG. 8 provides the results of cytotoxicity assays using CD19 CAR2-63 transduced T cells or non-transduced T cells from either PBMCs or cord blood (CB). Non-transduced control and CD19 CAR transduced T cells were incubated with CD19-expressing K562 tumor cells for 2 days at the indicated ratios.

FIG. 9 provides the results of cytotoxicity assays using CD19 CAR2-63 transduced T cells or non-transduced T cells from either PBMCs or cord blood (CB). Non-transduced control and CD19 CAR transduced T cells were incubated with CD19-expressing Raji tumor cells for 2 days at the indicated ratios.

FIG. 10 provides in vivo imaging of mice inoculated with tumor cells and then untreated or treated with T cells or transduced with CD19 CAR2-63 or as controls not transduced with a CAR. Immunocompromised NSG mice were inoculated intravenously with labeled Raji (CD19-expressing) tumor cells. Mice were intravenously treated with different doses of CD19 CAR-T cells where the T cells were either pan T cells or gd T cells, or as controls mice were untreated or were treated with T cells not transduced with the CD19 CAR construct. Tumor burden was assessed weekly by bioluminescent imaging (IVIS). For T cell-treated mice, T cells expressing the anti-CD19-CAR2-63 construct were a more effective treatment than non-transduced T cells, with pan T cells expressing the anti-CD19-CAR2-63 construct delivered at a dose of 10⁷ cells providing the greatest benefit.

FIG. 11 provides a graph of the tumor size based on the in vivo imaging of mice inoculated with tumor cells and then untreated or treated with gd or pan T cells not transduced with a CAR or transduce with CD19 CAR2-63. Also shown is a graph providing the body weight change of mice over the course of treatment.

FIG. 12 provides survivorship curves of mice inoculated with tumor cells and left untreated or treated with CD19 CAR-T cells or non-transduced T cells.

FIG. 13 provides survivorship curves of mice inoculated with tumor cells and left untreated or treated with CD19 CAR-T cells or non-transduced T cells.

### DETAILED DESCRIPTION

The disclosed chimeric antigen receptor (CAR) constructs preferentially bind to tumor cells that express CD19. Transformed T cells are provided that include the CD19 CAR constructs that encode an scFv antibody having a light chain variable (VL) domain comprising an amino acid sequence that is at least 95% homologous to the amino acid sequence of SEQ ID NO:1 and a heavy chain variable (VH) domain having an amino acid sequence that is at least 95% homologous to the amino acid sequence of SEQ ID NO:2.

### Definitions

Unless defined otherwise, technical and scientific terms used herein have meanings that are commonly understood by those of ordinary skill in the art unless defined otherwise. Generally, terminologies pertaining to techniques of cell and tissue culture, molecular biology, immunology, microbiology, genetics, transgenic cell production, protein chemistry and nucleic acid chemistry and hybridization described herein are well known and commonly used in the art.

The methods and techniques provided herein are generally performed according to conventional procedures well known in the art and as described in various general and more specific references that are cited and discussed herein unless otherwise indicated.

Throughout this application various publications, patents, and/or patent applications are referenced. The disclosures of the publications, patents and/or patent applications are hereby incorporated by reference in their entireties into this application in order to more fully describe the state of the art to which this disclosure pertains.

The headings provided herein are not limitations of the various aspects of the disclosure, which aspects can be understood by reference to the specification as a whole.

Unless otherwise required by context herein, singular terms shall include pluralities and plural terms shall include the singular. Singular forms “a”, “an” and “the”, and singular use of any word, include plural referents unless expressly and unequivocally limited on one referent.

It is understood the use of the alternative (e.g., “or”) herein is taken to mean either one or both or any combination thereof of the alternatives.

The term “and/or” used herein is to be taken mean specific disclosure of each of the specified features or components with or without the other. For example, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, terms “comprising”, “including”, “having” and “containing”, and their grammatical variants, as used herein are intended to be non-limiting so that one item or multiple items in a list do not exclude other items that can be substituted or added to the listed items. It is understood that wherever aspects are described herein with the language “comprising,” otherwise analogous aspects described in terms of “consisting of” and/or “consisting essentially of” are also provided.

The term “about” or “approximately” means an acceptable error for a particular value as determined by one of ordinary skill in the art, which depends, in part, on how the value is measured or determined. For example, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

The term “isolated” refers to a protein (e.g., an antibody) or polynucleotide that is substantially free of other cellular material. A protein may be rendered substantially free of naturally associated components (or components associated with the cellular expression system used to produce the antibody) by isolation, using protein purification techniques well known in the art. Isolated cells are removed from their in vivo cellular milieu, for example, are no longer associated with the tissue they are associated with in vivo (e.g., blood, tumor) and may be partially or substantially isolated from or enriched with respect to other cell types present in the tissue they are associated with in vivo. In various embodiments, constructs of the disclosure that encode CD19 chimeric antigen receptors, or host cells that include and/or express the disclosed constructs encoding the CD19 chimeric antigen receptors, are isolated.

The terms “nucleic acid”, “polynucleotide” and “oligonucleotide” and are used interchangeably and refers to polymers of nucleotides. Nucleic acids include naturally-occurring and recombinant forms. Nucleic acids include DNA molecules (cDNA or genomic DNA), RNA molecules (e.g., mRNA), analogs of the DNA or RNA generated using nucleotide analogs (e.g., peptide nucleic acids and non-naturally occurring nucleotide analogs), and hybrids thereof. Nucleic acid molecule can be single-stranded or double-stranded. In one embodiment, the nucleic acid molecules of the disclosure comprise a contiguous open reading frame encoding an antibody, or a fragment or scFv, derivative, mutein, or variant thereof.

The terms “peptide”, “polypeptide” and “protein” are used interchangeably and refer to a polymer of amino acids and are not limited to any particular length. Polypeptides comprise natural and non-natural amino acids. Polypeptides can be naturally-occurring or recombinant forms. These terms encompass native and artificial proteins, protein fragments and polypeptide analogs (such as muteins, variants, chimeric proteins and fusion proteins) of a protein sequence as well as post-translationally, or otherwise covalently or non-covalently, modified proteins. A peptide, polypeptide, or protein may be monomeric or polymeric. Polypeptides includes antibodies, antibody chains, scFv and chimeric antigen receptor constructs.

The “percent identity” or “percent homology” refers to a quantitative measurement of the similarity between two polypeptide or between two polynucleotide sequences. The percent identity between two polypeptide sequences is a function of the number of identical amino acids at aligned positions that are shared between the two polypeptide sequences, taking into account the number of gaps, and the length of each gap, which may need to be introduced to optimize alignment of the two polypeptide sequences. In a similar manner, the percent identity between two polynucleotide sequences is a function of the number of identical nucleotides at aligned positions that are shared between the two polynucleotide sequences, taking into account the number of gaps, and the length of each gap, which may need to be introduced to optimize alignment of the two polynucleotide sequences. A comparison of the sequences and determination of the percent identity between two polypeptide sequences, or between two polynucleotide sequences, may be accomplished using a mathematical algorithm. For example, the “percent identity” or “percent homology” of two polypeptide or two polynucleotide sequences may be determined by comparing the sequences using the GAP computer program (a part of the GCG Wisconsin Package, version 10.3 (Accelrys, San Diego, Calif.)) using its default parameters.

The terms “Chimeric Antigen Receptor” or “CAR” describes a fusion protein comprising an extracellular antigen-binding protein, preferably a single chain variable fragment (scFv or sFv) derived from fusing the variable heavy and light regions of a monoclonal antibody, that is fused to an intracellular signaling domain capable of activating or stimulating an immune cell. Alternatively, scFvs may be used that are derived from Fab's (instead of from an antibody, e.g., obtained from Fab libraries). Disclosed herein are CD19 CARs that include targeting moieties that specifically bind CD19.

The term “antibody” describes an immunoglobulin (lg) molecule comprised of four polypeptide chains, two heavy (H) chains and two light (L) chains, or any functional fragment, mutant, variant, or derivation thereof, which retains the essential epitope binding features of an Ig molecule. The term antibody includes, for example, single chain variable fragment antibodies (scFvs).

The terms “anti-CD19 antibody” and “an antibody that binds to CD19” refer to an antibody that is capable of specifically binding CD19.

A “single-chain antibody” or “scFv” or “single chain variable fragment [antibody]” is an antibody in which a V_{L} and a V_{H} region are joined via a linker (e.g., a synthetic sequence of amino acid residues) to form a continuous protein chain. In one embodiment, the linker is long enough to allow the protein chain to fold back on itself and form a monovalent antigen binding site (see, e.g., Bird et al., 1988, Science 242:423-26 and Huston et al., 1988, Proc. Natl. Acad. Sci. USA 85:5879-83).

A Fab fragment is a monovalent fragment having the V_{L}, V_{H}, C_{L} and C_{H1} domains; a F(ab′)₂ fragment is a bivalent fragment having two Fab fragments linked by a disulfide bridge at the hinge region; a Fd fragment has the VH and CHI domains; an Fv fragment has the V_{L} and V_{H} domains of a single arm of an antibody; and a dAb fragment has a V_{H} domain, a V_{L} domain, or an antigen-binding fragment of a V_{H} or V_{L} domain (U.S. Pat. Nos. 6,846,634 and 6,696,245).

The term “hinge” refers to an amino acid segment that is generally found between two domains of a protein and may allow for flexibility of the overall construct and movement of one or both of the domains relative to one another. Structurally, a hinge region typically comprises from about 8 to about 100 amino acids, e.g., from about 9 to about 75 amino acids, from about 10 to about 70 amino acids, or from about 20 to about 65 amino acids. In various embodiments, a hinge region is 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 100 amino acids in length. The hinge region of a CAR can be derived from is a hinge region of a naturally-occurring protein, such as a CD8 hinge region, a CD8α hinge region, a CD28 hinge region, a CD3ζ hinge region, a CD4 hinge region, a hinge region of an antibody (e.g., an IgG, IgA, IgM, IgE, or IgD antibody), or a hinge region that joins the constant domains CH1 and CH2 of an antibody or a fragment of any thereof. The hinge region can be derived from an immunoglobulin superfamily member or an antibody and may or may not comprise one or more constant regions of an antibody. A hinge region can comprise the hinge region of an antibody and the CH3 constant region of the antibody, or a hinge region can comprise the hinge region of an antibody and the CH2 and CH3 constant regions of the antibody. A hinge region can also be a non-naturally occurring peptide. A hinge region can be positioned between the C-terminus of the scFv moiety of a CAR and the N-terminus of the transmembrane domain of the CAR. In some embodiments, the hinge region comprises any one or any combination of two or more regions comprising an upper, core, or lower hinge sequence from an IgG1, IgG2, IgG3 or lgG4 immunoglobulin molecule. In some embodiments, a hinge region comprises an IgG1 upper hinge sequence EPKSCDKTHT (SEQ ID NO:17). In some embodiments, the hinge region comprises an IgG1 core hinge sequence CPXC, wherein X is P, R or S. In some embodiments, the hinge region comprises a lower hinge/CH2 sequence PAPELLGGP (SEQ ID NO:18). In some embodiments, the hinge is joined to an Fc region (CH2) having the amino acid sequence SVFLFPPKPKDT (SEQ ID NO:19). In one embodiment, the hinge region includes the amino acid sequence of an upper, core and lower hinge and comprises EPKSCDKTHTCPPCPAPELLGGP (SEQ ID NO:20). In some embodiments, the hinge region comprises one, two, three or more cysteines that can form at least one, two, three or more interchain disulfide bonds.

A “vector” refers to a nucleic acid molecule (e.g., DNA or RNA) which can be operably linked to foreign genetic material (e.g., nucleic acid transgene). Vectors can be single-stranded or double-stranded nucleic acid molecules. Vectors can be linear or circular nucleic acid molecules. Vectors can be used as a vehicle to introduce foreign genetic material into a cell (e.g., host cell). One type of vector is a “plasmid,” which refers to a linear or circular double stranded extrachromosomal DNA molecule which can be linked to a transgene, and is capable of replicating in a host cell, and transcribing and translating the transgene. A viral vector typically contains viral RNA or DNA backbone sequences which can be linked to the transgene. The viral backbone sequences can be modified to disable infection but retain insertion of the viral backbone and the co-linked transgene into a host cell genome. Examples of viral vectors include retroviral, lentiviral and adenoviral vectors. Certain vectors are capable of autonomous replication in a host cell into which they are introduced (e.g., bacterial vectors comprising a bacterial origin of replication and episomal mammalian vectors). Other vectors (e.g., non-episomal mammalian vectors) are integrated into the genome of a host cell upon introduction into the host cell, and thereby are replicated along with the host genome. An “expression vector” is a type of vector that can contain one or more regulatory sequences, such as inducible and/or constitutive promoters, or ribosomal binding sites, which directs transcription, or transcription and translation, of a transgene linked to the expression vector which is transduced into a host cell.

A transgene is “operably linked” to a vector when there is linkage between the transgene and the vector to permit functioning or expression of the vector sequences contained in the vector. Vector sequences can any one or any combination of an original-of-replication sequence, an inducible or constitutive promoter or enhancer sequence, at least one selectable marker sequence, 5′ and 3′ LTR sequences, and optionally viral env, pol and/or gag sequences.

A transgene is “operably linked” to a regulatory sequence when the regulatory sequence affects the expression (e.g., the level, timing, or location of expression) of the transgene. A “regulatory sequence” is a nucleic acid sequence that affects the expression (e.g., the level, timing, or location of expression) of a transgene to which it is operably linked. The regulatory sequence can, for example, exert its effects directly on the regulated nucleic acid, or through the action of one or more other molecules (e.g., polypeptides that bind to the regulatory sequence and/or the nucleic acid). Regulatory sequences can be part of a vector. Examples of regulatory sequences include promoters, enhancers, ribosomal binding sites and other expression control elements (e.g., polyadenylation signals). Further examples of regulatory sequences are described in, for example, Goeddel, 1990, Gene Expression Technology: Methods in Enzymology 185, Academic Press, San Diego, Calif. and Baron et al., 1995, Nucleic Acids Res. 23:3605-3606.

A “host cell” or “or a population of host cells” refers to a cell (or a population thereof) into which foreign (exogenous) nucleic acids have been introduced. The foreign nucleic acids can include an expression vector operably linked to a transgene, and the host cell can be used to express the foreign nucleic acid (transgene). In one example, the host cell (or population thereof) can be introduced with an expression vector operably linked to a nucleic acid encoding the chimeric antigen receptors (CAR) described herein. A host cell (or a population thereof) can be a cultured cell or can be extracted from a subject. The host cell (or a population thereof) includes the primary subject cell and its progeny without any regard for the number of passages. Progeny cells may or may not harbor identical genetic material compared to the parent cell. Host cells encompass progeny cells.

A host cell can be a prokaryotic cell, for example, E. coli, or it can be a eukaryotic cell, for example, a single-celled eukaryote (e.g., a yeast or other fungus), a plant cell (e.g., a tobacco or tomato plant cell), an animal cell (e.g., a human cell, a monkey cell, a hamster cell, a rat cell, a mouse cell, or an insect cell) or a hybridoma. Examples of host cells include RPMI8226 (Gentry et al., 2004 Leuk. Res. 28(3):307-313), and human chronic myelogenous leukemia cell line K562. Other examples include the COS-7 line of monkey kidney cells (ATCC CRL 1651) (Gluzman et al., 1981, Cell 23:175), L cells, C127 cells, 3T3 cells (ATCC CCL 163), Chinese hamster ovary (CHO) cells or their derivatives such as Veggie CHO and related cell lines which grow in serum-free media (Rasmussen et al., 1998, Cytotechnology 28:31) or CHO strain DX-B11, which is deficient in DHFR (Urlaub et al., 1980, Proc. Natl. Acad. Sci. USA 77:4216-20), HeLa cells, BHK (ATCC CRL 10) cell lines, the CV1/EBNA cell line derived from the African green monkey kidney cell line CV1 (ATCC CCL 70) (McMahan et al., 1991, EMBO J. 10:2821), human embryonic kidney cells such as 293, 293 EBNA or MSR 293, human epidermal A431 cells, human Colo205 cells, other transformed primate cell lines, normal diploid cells, cell strains derived from in vitro culture of primary tissue, primary explants, HL-60, U937, HaK or Jurkat cells. In one embodiment, a host cell is a mammalian host cell, for example a human host cell. Typically, a host cell is primary cell or a cultured cell that can be introduced with an exogenous polypeptide-encoding nucleic acid which can then be expressed in the host cell. It is understood that the term host cell refers to the particular subject cell and also to the progeny or potential progeny of such a cell. Because certain modifications may occur in succeeding generations due to, e.g., mutation or environmental influence, such progeny may not, in fact, be identical to the parent cell, but are still included within the scope of the term as used herein.

A host cell describes any cell (including its progeny) that has been modified, transfected, transduced, transformed, and/or manipulated in any way to express an anti-CD19-CAR construct, as disclosed herein. Preferably, the host cell is a human T cell, placenta cell or NK cell.

“Pan T cells” refers to a population of T cells that have not been selected by T cell type, but include all of the T cell types of the biological sample from which they are obtained, for example, pan T cells may include CD4+ “helper” T cells, CD4+CD25+ regulatory T cells, CD8+ cytotoxic T cells, gamma delta (γδ or gd) T cells, Natural Killer (NK) T cells, and “double negative” T cells. The biological source can be, as nonlimiting examples, peripheral blood, PBMCs, cord blood (CB), or placental blood, as nonlimiting examples.

As used herein, “allogeneic” refers to any material (such as cells) derived from a different individual of the same species as the individual to whom the material is introduced. Two or more individuals are said to be allogeneic to one another when the genes at one or more loci are not identical.

The terms “transfected” or “transformed” or “transduced” refer to a process by which exogenous nucleic acid (e.g., transgene) is transferred or introduced into a host cell. The term “transduced” is used to refer to introduction of nucleic acid into a host cell by means of a viral vector, such as by viral infection of the host cell. A “transfected”, “transformed” or “transduced” host cell is a cell which has been transfected, transformed, or transduced with exogenous nucleic acid or is the progeny of the directly transformed, transfected, or transduced cell.

Transgenes, such as the disclosed nucleic acid sequences encoding CD19 chimeric antigen receptors (CAR) constructs can be operably linked to a vector, including a viral vector, which is used as a vehicle to introduce a transgene into a host cell. Transgenes introduced (e.g., via transduction, transfection or transformation) into host cells can be transiently introduced or preferably stably integrated into the host cell's genome. Transgenes introduced into host cells can be propagated in progeny cells. Vectors can be single- or double-stranded DNA or RNA vectors. Vectors include expression vectors which direct expression of transgenes in a host cell. Suitable vectors include expression vectors which can contain an original of replication sequence, an inducible or constitutive promoter sequence, and at least one selectable marker sequence, where these sequences are functional in a packaging cell and/or host cell. Viral vectors used to introduce transgene into a host cell include vectors derived from the viral family Retroviridae which includes retroviral and lentiviral vectors. Retroviral vectors can be used to transduce dividing host cells, and lentiviral vectors can be used to transduce non-dividing host cells. Host cells transduced with the desired transgene linked to an expression vector include T cells, placental derived natural killer host cells, and cord blood derived natural killer host cells.

Retroviral vectors can be derived from any avian or mammalian source. Retroviral vectors can be capable of infecting host cells of several different species (e.g., amphotropic), including mice, rats and humans, or can have limited host range (e.g., ecotropic). Retroviral vectors can be derived from Moloney murine leukemia virus (MoMLV) (e.g., the MFG vector or a derivative thereof, Riviere et al. (1995) Proc. Nat'l. Acad. of Sci. USA 92:6733-6737), myeloproliferative sarcoma virus (MPSV), murine embryonic stem cell virus (MESV), murine stem cell virus (MSCV), spleen focus forming virus (SFFV).

In a typical first generation retroviral transfer vector (e.g., gammaretroviral vector) system, sequences that encode retroviral gag, pol and env can be replaced with a desired transgene, and the transgene can be flanked on both sides by cis-acting long terminal repeat (LTR) sequences. The gag and pol sequences can be carried on a packaging plasmid, the env sequence can be carried separately on an envelope plasmid, and expression of these three viral sequences act in-trans. The transfer vector (containing the transgene) along with the packaging and envelope plasmids, are reacted with packaging cells in the presence of a transfection reagent to transduce the vector and plasmids into the packaging cells. The transduced packaging cells produce cell culture supernatant containing infectious virions harboring the transfer vector carrying the transgene. Transduced host cells are generated by reacting the host cells with the virion supernatant. Upon transduction the retroviral transfer vector (carrying the transgene) integrates into the host cell's genome (Morgan and Boyerinas 2016 Biomedicines 4(2):9 “Review: Genetic Modification of T Cells”). Retroviral transfer vectors can also contain a promoter that directs inducible or constitutive transcription of the transgene. A second generation retroviral vector system typically includes gag, pol and env sequence stably expressed in a packaging cell line which obviates the need for separate packaging and envelope plasmids. The packaging cell line is reacted with the packaging vector (carrying the transgene) to generate transduced packaging cells and virion supernatant. Phoenix helper-free retroviral packaging cell lines is an example of a second generation retroviral system. Retroviral vectors are used for host cell transduction (WO2014/055668).

Lentivirus vectors derived from HIV, SIV or FIV, can be used to introduce a transgene into a host cell. Several generations of lentivirus vectors have been developed. First generation lentiviral systems are similar to first generation retroviral systems in that they employ a transfer vector (carrying the transgene), packaging plasmid (carrying gag, pol, tat, rev and accessory sequences), and envelope plasmid (carrying a heterologous env sequence). Second generation lentiviral systems employ a transfer vector (transgene), packaging plasmid (gag, pol, tat and rev, and accessory sequences removed), and envelope plasmid (carrying a heterologous env sequence). Third generation lentiviral systems, sometimes called self-inactivating (SIN) systems, employ a transfer vector (transgene and 3′ LTR having tat removed), a first packaging plasmid (gag and pol), a second packaging plasmid (rev), and envelope plasmid (carrying a heterologous env sequence). Similar to retroviral systems, any of these lentiviral systems involves reacting the vector/plasmids with packaging cells and a transduction reagent to produce cell culture supernatant containing virions which is in turn used to transduce host cells. Lentivirus vectors are used to transduce host cells (WO2012/031744; U.S. Pat. No. 8,802,374; and U.S. 2016/0152723).

Other viral vectors used to introduce transgenes into host cells include simian virus 40 (SV40), herpes simplex virus 1, adenovirus, adeno-associated virus (AAV) and Rous sarcoma virus (RSV) (Gross 1989 Proc. Natl. Acad. Sci. USA 86:10024-10028).

Expression vectors typically include a promoter and/or enhancer sequence that directs inducible or constitutive expression (e.g., transcription) in packaging cells and/or host cells to be introduced with a transgene. Constitutive promoters include retroviral LTR, immediate early cytomegalovirus (CMV) promoter, elongation growth factor 1 alpha (EF-1α), simian virus 40 (SV40) early promoter, mouse mammary tumor virus (MMTV) promoter, human immunodeficiency virus (HIV) long terminal repeat (LTR) promoter, Moloney murine leukemia virus (MoMuLV) LTR promoter, avian leukemia virus promoter, Epstein-Barr virus immediate early promoter, Rous sarcoma virus promoter, PGK (phosphoglycerate kinase), UbC (Ubiquitin C), MLV (Moloney leukemia virus) and CAG (cytomegalovirus early enhancer element, promoter from first exon and intron of chicken beta-actin, and splice acceptor of rabbit beta-globin) enhancer sequence. Inducible promoter sequences include tetracycline operator (TetO) sites (Sakemura 2016 Cancer Immunology Research 4(8):658-668) and lac repressor system from E. coli. Promoters suitable for high expression from lentiviral vectors include human ubiquitin, MHC class I, MHC class II, and β2 microglobulin promoters (WO 2016/012623). Retroviral and lentiviral expression vectors are commercially available from several sources including Applied Biological Materials (ABM) (Vancouver, Canada) and Addgene (Watertown, Massachusetts).

The term “target cells” are cells expressing one or more target polypeptides which renders them recognizable by an antibody or antibody derivative. In one embodiment, target cells include cancer (tumor) target cells expressing CD19 polypeptides which are recognized for binding by chimeric antigen receptor (CAR) constructs of the present disclosure.

### CD19 Chimeric Antigen Receptors (CD19 CARs)

The present disclosure describes a new CAR construct comprising an anti-CD19 antibody onto a second-generation CAR construct scaffold and generally with different component transmembrane domains and intracellular domains. The present disclosure provides a nucleic acid sequence encoding a CD19 CAR for transduction into T cells, including pan T cells, T cells isolated from placental tissue or cord blood, or gamma delta (gd) T cells, in which the CAR directs the T cells to CD19-expressing tumor cells.

CARs are generally constructed by joining the antigen recognition domains of an antibody with the signaling domains of receptors from T cells. A CAR construct will typically contain sequences encoding an extracellular region, e.g., a single chain variable fragment (scFv) of an antibody recognizing an antigen present on cancer cells (such as CD19), sequences encoding an intracellular region, e.g., a T-cell receptor such as (TCR) zeta chain that mimics TCR activation, and sequences encoding at least one signaling domain derived from CD28 or 4-ABB to mimic co-stimulation. Modification of T cells with nucleic acid sequences encoding CARs equips T cells with retargeted antibody-type antitumor cytotoxicity. Because killing is MHC-unrestricted, the approach offers a general therapy for all patients bearing the same antigen. These T cells engineered with CARs are often called “CAR-T cells”, “designer T cells”, or “T-bodies” (Eshhar et al. Proc. Natl. Acad. Sci. USA 90(2): 720-724,1993; Ma et al., Cancer Chemother. Bio. I Response Modif. 20: 315-341, 2002).

The anti-CD19 antibody light chain can comprise the amino acid sequence of SEQ ID NO: 1 or an amino acid sequence having at least 95%, 96%, 97%, 98%, or 99% identity to SEQ ID NO: 1 and the heavy chain can comprise the amino acid sequence SEQ ID NO:2 or an amino acid sequence having at least 95%, 96%, 97%, 98%, or 99% identity to SEQ ID NO:2. An anti-CD19 scFv antibody can comprise the amino acid sequence SEQ ID NO: 12 or an amino acid sequence having at least 95%, 96%, 97%, 98%, or 99% identity to SEQ ID NO:12.

The disclosed CD19 CAR further comprises a hinge region, that preferably includes a CD8 hinge region (e.g., SEQ ID NO:4), a sequence having at least 95%, 96%, 97%, 98%, or 99% identity to SEQ ID NO:4, or a functional fragment of SEQ ID NO:4. The disclosed CD19 CAR preferably further comprises a further extracellular domain, preferably a CD28 extracellular domain or hinge region (SEQ ID NO:5), a hinge region having at least 95%, 96%, 97%, 98%, or 99% identity to SEQ ID NO:5, or a functional fragment thereof. The entire hinge region can have a length of at least 65, 70, 75, 80, or 85 amino acids, and can have at least 95%, 96%, 97%, 98%, or 99% identity to SEQ ID NO:13.

The disclosed CD19 CAR further comprises a transmembrane domain, preferably a transmembrane domain from the transmembrane domains of the protein selected from the group consisting of alpha chain of T-cell receptor, beta chain of T-cell receptor, zeta chain of T-cell receptor, CD28, CD3 epsilon, CD45, CD4, CD5, CD8, CD9, CD16, CD22, CD33, CD37, CD64, CD80, CD86, CD134, CD137, CD154, LFA-1 T-cell co-receptor, CD2 T-cell co-receptor/adhesion molecule, CD8 alpha, and combinations thereof. Preferably, the transmembrane domain is from CD28 transmembrane domain (SEQ ID NO:6), or a functional fragment thereof.

The disclosed CD19 CAR further comprises an intracellular signaling domain comprising signaling domains from the group consisting of a CD3-zeta chain, 4-1BB, CD28, and combination thereof. If there are two signaling domains, the second one is called a co-stimulatory signaling domain. Preferably, the co-stimulatory signaling domain comprises an intracellular domain, or fragment thereof, of, but not limited to, the following proteins: CD27, CD28, 4-1BB, OX40, CD30, CD40, PD-1, ICOS, lymphocyte function-associated antigen-1 (LFA-1), CD2, CD7, LIGHT, NKG2C, B7-H3, a CD83 ligand, and any combination thereof. In some embodiments, the intracellular signaling domain comprises a CD28 signaling domain. In further embodiments, the CD28 signaling domain comprises the amino acid sequence of SEQ ID NO:7, or a functional fragment thereof. In some embodiments, the intracellular signaling domain comprises a CD3-ζ signaling domain. In further embodiments, the CD3-ζ signaling domain comprises the amino acid sequence of SEQ ID NO:8, or a functional fragment thereof.

Thus, the present disclosure encompasses isolated nucleic acid molecules comprising sequences encoding the disclosed CD19 CAR construct. It should be noted that where an amino acid sequence is described, also included is a nucleic acid sequence that encodes the amino acid sequence.

### 1. Extracellular Anti-CD19 Binding Protein

The present disclosure provides a CD19 CAR comprising an antigen binding protein that binds to CD19, wherein the antigen binding protein comprises a light chain variable (VL) domain comprising an amino acid sequence that is at least 95% identical to the amino acid sequence of SEQ ID NO: 1. Preferably, the VL domain comprises an amino acid sequence that is at least 96% homologous to the amino acid sequence of SEQ ID NO: 1, or the VL domain comprises an amino acid sequence that is at least 97% identical to the amino acid sequence of SEQ ID NO:1, or the VL domain comprises an amino acid sequence that is at least 98% identical to the amino acid sequence of SEQ ID NO: 1, or the VL domain comprises an amino acid sequence that is at least 99% identical to the amino acid sequence of SEQ ID NO: 1.

The VL and VH domains can be joined in an scFv by a linker, such as but not limited to a GS (G₄S) linker, that can have, for example, between one and eight repeating units of G₄S. In some examples the linker has three G₄S units.

Further, the present disclosure provides a CAR comprising an antigen binding protein that binds to CD19, wherein the antigen binding protein comprises a heavy chain variable (VH) domain comprising an amino acid sequence that is at least 95% identical to the amino acid sequence of SEQ ID NO:2, or VH domain comprises an amino acid sequence that is at least 96% identical to the amino acid sequence of SEQ ID NO:2, or the VH domain comprises an amino acid sequence that is at least 97% identical to the amino acid sequence of SEQ ID NO:2, or the VH domain comprises an 2, or the VH domain comprises an amino acid sequence that is at least 99% identical to the amino acid sequence of SEQ ID NO:2. In some preferred embodiments, the disclosed CD19 CAR comprises an scFv, comprising a light chain having a variable domain comprising an amino acid sequence of SEQ ID NO:1; and a heavy chain having a variable domain comprising an amino acid sequence of SEQ ID NO:2.

In some embodiments provided herein a CD19 CAR has one or more variations, including amino acid substitutions, deletions and/or insertions, compared to the amino acid sequence of the CAR constructs described herein, so long as the variant CAR comprises an amino acid sequence that is at least 95% identical to SEQ ID NO:14.

Preferably, the disclosed CD19 CAR comprises an scFv, comprising a light chain having a variable domain comprising an amino acid sequence of SEQ ID NO: 1 and a heavy chain having a variable domain comprising an amino acid sequence of SEQ ID NO:2.

Single chain antibodies may be formed by linking heavy and light chain variable domain (Fv region) fragments via an amino acid bridge (short peptide linker), resulting in a single polypeptide chain. Such single-chain Fvs (scFvs) have been prepared by fusing DNA encoding a peptide linker between DNAs encoding the two variable domain polypeptides (VL and VH). The resulting polypeptides can fold back on themselves to form antigen-binding monomers, or they can form multimers (e.g., dimers, trimers, or tetramers), depending on the length of a flexible linker between the two variable domains (Kortt et al., 1997, Prot. Eng. 10:423; Kortt et al., 2001, Biomol. Eng. 18:95-108). By combining different VL- and VH-comprising polypeptides, one can form multimeric scFvs that bind to different epitopes (Kriangkum et al., 2001, Biomol. Eng. 18:31-40). Techniques developed for the production of single chain antibodies include those described in U.S. Pat. No. 4,946,778; Bird, 1988, Science 242:423; Huston et al., 1988, Proc. Natl. Acad. Sci. USA 85:5879; Ward et al., 1989, Nature 334:544, and de Graaf et al., 2002, Methods Mol. Biol. 178:379-87. One example of a linker that can join VL and VH regions of an scFv in a CD19 CAR as provided herein is the linker of SEQ ID NO:3.

### 2. Transmembrane Domains

A transmembrane domain of the disclosed CD19 CAR construct describes any polypeptide structure that is thermodynamically stable in a cell membrane, preferably a mammalian cell membrane. Transmembrane domains compatible for use in the disclosed CD19 CAR construct may be obtained from any natural transmembrane protein, or a fragment thereof. Alternatively, the transmembrane domain can be a synthetic, non-naturally occurring transmembrane protein, or a fragment thereof, e.g., a hydrophobic protein segment that is thermodynamically stable in a cell membrane (e.g., a mammalian cell membrane).

Preferably, the transmembrane domain used in a CAR is derived from a membrane protein selected from the group consisting of CD8α, CD8β, 4-1BB/CD137, CD28, CD34, CD4, FcεRIγ, CD16, OX40/CD134, CD3ζ, CD3ε, CD3γ, CD3δ, TCRα, TCRβ, TCRζ, CD32, CD64, CD64, CD45, CD5, CD9, CD22, CD33, CD37, CD64, CD80, CD86, CD137, CD154, LFA-1 T cell co-receptor, CD2 T cell co-receptor/adhesion molecule, CD40, CD4OL/CD154, VEGFR2, FAS, and FGFR2B. Preferably, the transmembrane domain is derived from CD8α, 4-1BB/CD137, CD28, or CD34.

In various embodiments provided herein the transmembrane domain of a CD19 CAR comprises an amino acid sequence derived from CD28, for example, an amino acid sequence that is at least 95% identical to the amino acid sequence of SEQ ID NO:6, at least 96% identical to the amino acid sequence of SEQ ID NO:6, or at least 97% identical to the amino acid sequence of SEQ ID NO:6, or the transmembrane domain comprises an amino acid sequence that is at least 98% identical to the amino acid sequence of SEQ ID NO:6 or is at least 99% identical to the amino acid sequence of SEQ ID NO:6. In some embodiments of a CD19 CAR, the CAR comprises the amino acid sequence of SEQ ID NO:6.

### 3. Intracellular Domains

The CD19 CARs disclosed herein comprise at least one intracellular signaling domain. A signaling domain is generally responsible for activation of at least one of the normal effector functions of a cell. The term “effector function” describes a specialized function of a cell. For example, the effector function of a T cell or an NK cell can include a cytolytic activity or a helper activity. “Signaling domain” describes the portion of a protein which transduces the effector function signal and directs the cell to perform its specialized function. While usually the entire intracellular signaling domain can be employed, in many cases it is not necessary to use an entire chain or domain. To the extent that a truncated portion of the intracellular signaling domain is used, such truncated portion may be used in place of the intact domain as long as it transduces the effector function signal.

A primary signaling domain regulates primary activation of the TCR complex either in a stimulatory way or in an inhibitory way. Primary signaling domains that act in a stimulatory manner may contain signaling motifs which are known as immunoreceptor tyrosine-based activation motifs (ITAMs). Primary signaling domains containing ITAMs for use in the CD19 CARs include the signaling domains of TCR zeta (CD3 zeta), FcR gamma, FcR beta, CD3 gamma, CD3 delta, CD3 epsilon, CD5, CD22, CD79a, CD79b, and CD66d. Preferably, a primary signaling domain is CD3ζ or CD28.

In various embodiments, a CD19 CAR may further comprise a co-stimulatory signaling domain. Examples of co-stimulatory signaling domains for use in the chimeric receptors are cytoplasmic signaling domain of co-stimulatory proteins selected from the group consisting of members of the B7/CD28 family (B7-1/CD80, B7-2/CD86, B7-H1/PD-L1, B7-H2, B7-H3, B7-H4, B7-H6, B7-H7, BTLA/CD272, CD28, CTLA-4, Gi24/VISTA/B7-H5, ICOS/CD278, PD-1, PD-L2/B7-DC, and PDCD6); members of the TNF superfamily (4-1BB/TNFSF9/CD137, 4-1BB ligand/TNFSF9, BAFF/BLyS/TNFSF13B, BAFF R/TNFRSF13C, CD27/TNFRSF7, CD27 ligand/TNFSF7, CD30/TNFRSF8, CD30 ligand/TNFSF8, CD40/TNFRSF5, CD40/TNFSF5, CD40 ligand/TNFSF5, DR3/TNFRSF25, GITR/TNFRSF18, GITR ligand/TNFSF18, HVEM/TNFRSF14, LIGHT/TNFSF14, lymphotoxin-alpha/TNF-beta, OX40/TNFRSF4, OX40 ligand/TNFSF4, RELT/TNFRSF19L, TACI/TNFRSF13B, TLIA/TNFSF15, TNF-α, and TNF RII/TNFRSF1B); members of the interleukin-1 receptor/toll-like receptor (TLR) superfamily (TLR1, TLR2, TLR3, TLR4, TLR5, TLR6, TLR7, TLR8, TLR9, and TLR10); members of the SLAM family (2B4/CD244/SLAMF4, BLAME/SLAMF8, CD2, CD2F-10/SLAMF9, CD48/SLAMF2, CD58/LFA-3, CD84/SLAMF5, CD229/SLAMF3, CRACC/SLAMF7, NTB-A/SLAMF6, and SLAM/CD150); CD2, CD7, CD53, CD82/Kai-1, CD90/Thy1, CD96, CD160, CD200, CD300a/LMIR1, HLA Class I, HLA-DR, ikaros, integrin alpha 4/CD49d, integrin alpha 4 beta 1, integrin alpha 4 beta 7/LPAM-1, LAG-3, TCL1A, TCL1B, CRTAM, DAP10, DAP12, MYD88, TRIF, TIRAP, TRAF, Dectin-1/CLEC7A, DPPIV/CD26, EphB6, TIM-1/KIM-1/HAVCR, TIM-4, TSLP, TSLP R, lymphocyte function associated antigen-1 (LFA-1), and NKG2C. Preferably, the co-stimulatory domain comprises an intracellular domain of an activating receptor protein selected from the group consisting of α₄β₁ integrin, β₂ integrins (CD11a-CD18, CD11b-CD18, CD11b-CD18), CD226, CRTAM, CD27, NKp46, CD16, NKp30, NKp44, NKp80, NKG2D, KIR-S, CD100, CD94/NKG2C, CD94/NKG2E, NKG2D, PEN5, CEACAM1, BY55, CRACC, Ly9, CD84, NTBA, 2B4, SAP, DAP10, DAP12, EAT2, FcRγ, CD3ζ, and ERT. Preferably, the co-stimulatory domain comprises an intracellular domain of an inhibitory receptor protein selected from the group consisting of KIR-L, LILRB1, CD94/NKG2A, KLRG-1, NKR-P1A, TIGIT, CEACAM, SIGLEC 3, SIGLEC 7, SIGLEC9, and LAIR-1. Preferably, the co-stimulatory domain comprises an intracellular domain of a protein selected from the group consisting of CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83.

In various embodiments of a CD19 CAR provided herein the CD19 CAR includes a first signaling domain and a second signaling domain. For example, the first signaling domain of the CD19 CAR can comprise a signaling domain derived from CD28, e.g., SEQ ID NO:7. The first signaling domain can comprise an amino acid sequence that is at least 95% identical to the amino acid sequence of SEQ ID NO:7, at least 96% identical to the amino acid sequence of SEQ ID NO:7, or at least 97% identical to the amino acid sequence of SEQ ID NO:7, or the first signaling domain can comprise an amino acid sequence that is at least 98% or at least 99% identical to the amino acid sequence of SEQ ID NO:7. In some embodiments, the first signaling domain comprises the amino acid sequence of SEQ ID NO:7.

A CD19 CAR as provided herein that comprises a second signaling domain can, for example, comprise a signaling domain derived from CD3ζ, e.g., SEQ ID NO:8. The second signaling domain can comprise an amino acid sequence that is at least 95% identical to the amino acid sequence of SEQ ID NO:8, at least 96% identical to the amino acid sequence of SEQ ID NO:8, or at least 97% identical to the amino acid sequence of SEQ ID NO:8, or the second signaling domain can comprise an amino acid sequence that is at least 98% or at least 99% identical to the amino acid sequence of SEQ ID NO:8. In some embodiments, the second signaling domain comprises the amino acid sequence of SEQ ID NO:8.

### 4. Hinge Regions

The CD19 CAR can further comprise a hinge region. The hinge region is located between the scFv antibody region and the transmembrane domain. A hinge region is an amino acid segment that is generally found between two domains of a protein and allows for flexibility of the CD19 CAR and movement of one or both of the domains relative to one another. The hinge region can comprise from about 7 to about 120 amino acids, e.g., from about 8 to about 100 amino acids, from about 10 to about 90 amino acids, or from about 50 to about 90 amino acids. Preferably the hinge region comprises at least 65 amino acids, for example, at least 65, at least 70, at least 75, at least 80, or at least 85 amino acids. A hinge region can be a hinge region of a naturally occurring protein or derived from a hinge region of a naturally occurring protein and can be derived from hinge regions of more than one polypeptide. In various embodiments, the hinge region comprises a CD8 hinge region, for example, a CD8α hinge region or a sequence having at least 95% identity thereto and further comprises a CD28 hinge region or a sequence having at least 95% identity thereto. Preferably, the hinge region is disposed between the C-terminus of the scFv and the N-terminus of the transmembrane domain of the CAR.

Preferably the hinge region of the CD19 CAR comprises an amino acid sequence that is at least 95% identical to the amino acid sequence of SEQ ID NO:4. For example the hinge regions may comprise the amino acid sequence of SEQ ID NO:4. Preferably the hinge region of the CD19 CAR further comprises an amino acid sequence that is at least 95% identical to the amino acid sequence of SEQ ID NO:5 For example the hinge regions may comprise the amino acid sequence of SEQ ID NO:5. In some preferred embodiments the hinge region comprises the sequence of SEQ ID NO:13 or a sequence having at least 95% identity thereto.

### 5. Signal Peptides

Signal sequences are peptide sequences that target a polypeptide to the desired site in a cell, such as the secretory pathway of the cell, and will allow for integration and anchoring of the CD19 CAR into the lipid bilayer of the cellular membrane. A signal peptide encoded by a CD19 CAR construct can be positioned at the N-terminus of the encoded CAR and is operable in the cell type in which the CAR is to be expressed. Preferably, the signal peptide is cleaved during integration of the CAR into the cell membrane and is not present on the mature CAR expressed by the host cell.

In various examples the signal sequence of a CD19 CAR as provided herein is derived from a signal sequence of a protein of the immunoglobulin superfamily, and may be derived from, for example, an immunoglobulin heavy chain signal sequence, or a signal sequence of a molecule expressed on the cell membrane such as, for example, CD8α, CD28, or CD16. In some embodiments the signal sequence of an CD19 CAR as provided herein comprises an amino acid sequence that is at least 95% identical to the amino acid sequence of SEQ ID NO:9, or the signal sequence comprises an amino acid sequence that is at least 96% identical or 97% identical to the amino acid sequence of SEQ ID NO:9, or the signal sequence comprises an amino acid sequence that is at least 98% identical to the amino acid sequence of SEQ ID NO:9 or at least 99% identical to the amino acid sequence of SEQ ID NO:9. In exemplary embodiments the signal sequence comprises the amino acid sequence of SEQ ID NO:9.

### Host Cells

Isolated host cells or populations of host cells are transduced with a disclosed CD19 construct to express the CD19 CAR. The host cells may be transduced with a lentivirus or retrovirus, for example, as disclosed herein. In the alternative, host cells can be generated by CRISPR/Cas methods, for example, as disclosed in US 20200224160 and WO2020185867, incorporated herein by reference.

In some embodiments the host cells are patient-derived isolated T cells, i.e., autologous T cells. Such populations of autologous T cells are obtained from a patient (e.g., from peripheral blood which is processed to provide peripheral blood mononuclear cells (PBMCs) from which T cells are isolated), isolated, and expanded. The isolated and expanded T cells originating from the patient are then transduced with a CD19 CAR construct as disclosed herein to achieve a population of transduced CD19 CAR-T cells that are typically expanded and returned (administered) to the individual patient.

In other embodiments the host cells are cultured T cells of derived from placenta or cord blood (CB) after pregnancy. Such populations of T cells are obtained from tissue not derived from the patient (allogeneic). The T cells are isolated and then expanded. The expanded placental or CB T cells are then transduced with a CD19 CAR construct as disclosed herein to achieve a population of transduced CD19 CAR-T cells. The transduced CAR-T cells are expanded and administered to a patient unrelated to the source of the T cells.

The present disclosure provides cultured T cells transduced with a CD19 CAR construct as provided herein that express the CD19 CAR. The T cells may be isolated from PBMCs or may be isolated from placental tissue or cord blood (CB).

### Therapeutic Methods and Uses of CD19 CARs

The present disclosure provides methods for treating a cancer or inhibiting tumor growth in a subject in need thereof, the method comprising administering to the subject an isolated host cell comprising a CD19 CAR, or a population of transduced host cells. Hematologic cancer can be treated using the CD19 CARs disclosed herein. Examples of hematologic cancer that can be treated using the methods of the disclosure include non-Hodgkin's lymphoma (NHL), Burkitt's lymphoma (BL), B chronic lymphocytic leukemia (B-CLL), B acute lymphocytic leukemia (ALL), T cell lymphoma (TCL), T acute lymphocytic leukemia (ALL), acute myeloid leukemia (AML), hairy cell leukemia (HCL), Hodgkin's Lymphoma (HL), and chronic myeloid leukemia (CML). For example, a patient can be administered isolated CD19 CAR-T cells to treat non-Hodgkin's lymphoma (NHL), B chronic lymphocytic leukemia (B-CLL), or B acute lymphocytic leukemia (ALL).

The disclosure provides a method of inhibiting growth of a tumor expressing a cancer associated antigen, comprising contacting a cancer cell of the tumor with a transduced host cell comprising a CD19 CAR, or a population of transduced host cells, wherein the host cell is an autologous T cell or a placenta-derived or CB-derived T cell.

The transduced host cells may be administered at a dosage of about 10¹ to about 10⁹ cells/kg body weight. Ranges intermediate to the above recited dosage, e.g., about 10² to about 10⁸ cells/kg body weight, about 10⁴ to about 10⁷ cells/kg body weight, about 10⁵ to about 10⁶ cells/kg body weight, are also intended to be part of this disclosure.

The host cells administered to a subject or patient can be allogeneic with respect to the subject or patient.

The transduced host cells may be administered as a single dose or multiple doses, and may be administered daily or preferably less frequently.

### EXAMPLES

The following examples are meant to be illustrative and can be used to further understand embodiments of the present disclosure and should not be construed as limiting the scope of the present teachings in any way.

### Example 1: CD19 CAR2 Construct

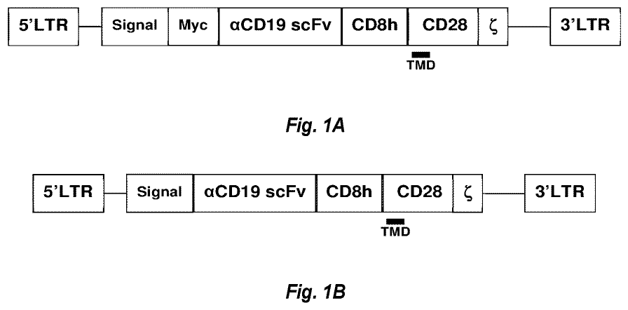

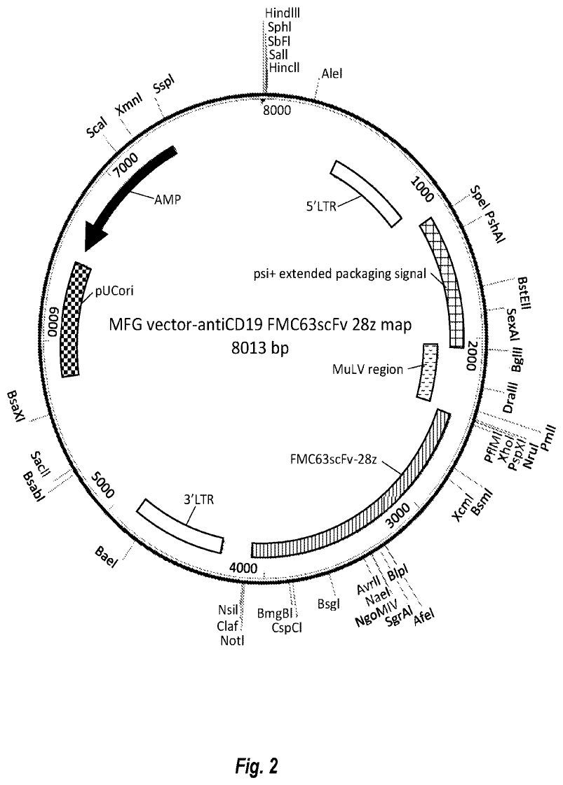

A CD19 CAR2 construct (schematically represented in FIG. 1A) was assembled by fusing in frame a sequence encoding a single chain variable fragment (scFv) antibody (SEQ ID NO:12) that included the light chain variable region of SEQ ID NO: 1 and the heavy chain variable region of SEQ ID NO:2 joined by the peptide linker sequence of SEQ ID NO:3. A sequence encoding a signal peptide (SEQ ID NO:9) was fused upstream of the anti-CD19 scFv-encoding sequence, and a sequence encoding a myc tag (SEQ ID NO: 10) was inserted between the signal peptide-encoding sequence and the anti-CD19 scFv-encoding sequence, preceded by the introduced amino acids aspartate and isoleucine (SEQ ID NO:11). Downstream of the anti-CD19 scFv-encoding sequence were sequences encoding, in order (N-terminal to C-terminal): a hinge region derived from CD8 (SEQ ID NO:4), an additional two amino acids (PR) as a linker, an extracellular hinge region derived from CD28 (SEQ ID NO:5), a transmembrane domain derived from CD28 (SEQ ID NO:6), an intracellular signaling domain derived from CD28 (SEQ ID NO:7), and a signaling domain derived from CD3ζ (SEQ ID NO:8). The amino acid sequence of the entire CD19 CAR2 precursor (with signal peptide), referred to herein as “CD19 CAR2-63” is provided as SEQ ID NO:14, and is encoded by the nucleic acid sequence of SEQ ID NO:15. The CD19 CAR2-63 construct was cloned into a retroviral vector to provide the retroviral vector construct shown in FIG. 2 (SEQ ID NO:16), where the 5′ LTR was used to provide the promoter for driving expression of the CD19 CAR. The mature polypeptide of the CD19 CAR2-63 is provided as SEQ ID NO:23.

A second construct was produced that was essentially identical to the first, except that this construct lacked the myc tag sequence (schematically represented in FIG. 1B). This construct included a sequence encoding a single chain variable fragment (scFv) antibody (SEQ ID NO:12) that included the light chain variable region of SEQ ID NO:1 and the heavy chain variable region of SEQ ID NO:2 joined by the peptide linker sequence of SEQ ID NO:3. The nucleic acid sequence encoding the anti-CD19 scFv (SEQ ID NO:12) was fused in frame to a sequence (proceeding N-terminal to C-terminal): a hinge region derived from CD8 (SEQ ID NO:4), an additional two amino acids (PR) as a linker, a hinge region derived from CD28 (SEQ ID NO:5), a transmembrane domain derived from CD28 (SEQ ID NO:6), an intracellular signaling domain derived from CD28 (SEQ ID NO:7), and a signaling domain derived from CDζ (SEQ ID NO:8). The construct also included a sequence encoding a signal peptide that was positioned at the N-terminus of the entire polypeptide. The amino acid sequence of the entire CD19 CAR2 precursor (with signal peptide and lacking a myc tag), referred to herein as “CD19 CAR2-63a” is provided as SEQ ID NO:24, and is encoded by the nucleic acid sequence of SEQ ID NO:25. The CD19 CAR2-63a construct was cloned into a retroviral vector to provide the retroviral vector construct shown schematically in FIG. 2 (SEQ ID NO:26), where the 5′ LTR was used to provide the promoter for driving expression of the CD19 CAR. The mature polypeptide of the CD19 CAR2-63a is provided as SEQ ID NO:27.

TABLE 1
Sequences

| SEQ ID | | |
|---|---|---|
| NO: | SEQUENCE | DESCRIPTION |
| 1 | DIQMTQTTSSLSASLGDRVTISCRASQDISKYLNWYQQKP | Anti-CD19 antibody, |
| | DGTVKLLIYHTSRLHSGVPSRFSGSGSGTDYSLTISNLEQ | variable light chain |
| | EDIATYFCQQGNTLPYTFGGGTKLEIT | |
| 2 | EVKLQESGPGLVAPSQSLSVTCTVSGVSLPDYGVSWIRQP | Anti-CD19 antibody, |
| | PRKGLEWLGVIWGSETTYYNSALKSRLTIIKDNSKSQVFL | variable heavy chain |
| | KMNSLQTDDTAIYYCAKHYYYGGSYAMDYWGQGISVTVSS | |
| 3 | GGGGSGGGGSGGGGS | peptide linker |
| | | connecting VH and |
| | | VL |
| 4 | AKPTTTPAPRPPTPAPTIASQPLSLRPEACRPAAGGAVHT | CD8α hinge region |
| | RGLDFA | |
| 5 | KIEVMYPPPYLDNEKSNGTIIHVKGKHLCPSPLFPGPSKP | CD28 hinge region |
| | | (extracellular domain/ |
| | | spacer) |
| 6 | FWVLVVVGGVLACYSLLVTVAFIIFWV | CD28 transmembrane |
| | | domain |
| 7 | RSKRSRLLHSDYMNMTPRRPGPTRKHYQPYAPPRDFAAYR | CD28 intracellular |
| | S | signaling domain |
| 8 | RVKFSRSADAPAYQQGQNQLYNELNLGRREEYDVLDKRRG | CD3-ζ signaling |
| | RDPEMGGKPQRRKNPQEGLYNELQKDKMAEAYSEIGMKGE | domain |
| | RRRGKGHDGLYQGLSTATKDTYDALHMQALPPR | |
| 9 | MEWSWVFLFFLSVTTGVHS | signal peptide from |
| | | mouse antibody heavy |
| | | chain |
| 10 | EQKLISEEDL | myc tag |
| 11 | DIEQKLISEEDL | myc tag with 2 |
| | | additional amino |
| | | acids |
| 12 | DIQMTQTTSSLSASLGDRVTISCRASQDISKYLNWYQQKP | CD19 scFv: |
| | DGTVKLLIYHTSRLHSGVPSRFSGSGSGTDYSLTISNLEQ | V_{L}-(G₄S)₃-V_{H} |
| | EDIATYFCQQGNTLPYTFGGGTKLEITGGGGSGGGGSGGG | |
| | GSEVKLQESGPGLVAPSQSLSVTCTVSGVSLPDYGVSWIR | |
| | QPPRKGLEWLGVIWGSETTYYNSALKSRLTIIKDNSKSQV | |
| | FLKMNSLQTDDTAIYYCAKHYYYGGSYAMDYWGQGTSVTV | |
| | SS | |
| 13 | AKPTTTPAPRPPTPAPTIASQPLSLRPEACRPAAGGAVHT | CD8 plus CD28 |
| | RGLDFAPRKIEVMYPPPYLDNEKSNGTIIHVKGKHLCPSP | Hinge sequence |
| | LFPGPSKP | |
| 14 | MEWSWVFLFFLSVTTGVHSDIEQKLISEEDLDIQMTQTTS | CD19 CAR2-63 |
| | SLSASLGDRVTISCRASQDISKYLNWYQQKPDGTVKLLIY | (precursor |
| | HTSRLHSGVPSRFSGSGSGTDYSLTISNLEQEDIATYFCQ | with signal |
| | QGNTLPYTFGGGTKLEITGGGGSGGGGSGGGGSEVKLQES | peptide) |
| | GPGLVAPSQSLSVTCTVSGVSLPDYGVSWIRQPPRKGLEW | |
| | LGVIWGSETTYYNSALKSRLTIIKDNSKSQVFLKMNSLOT | |
| | DDTAIYYCAKHYYYGGSYAMDYWGQGTSVTVSSAKPTTTP | |
| | APRPPTPAPTIASQPLSLRPEACRPAAGGAVHTRGLDFAP | |
| | RKIEVMYPPPYLDNEKSNGTIIHVKGKHLCPSPLFPGPSK | |
| | PFWVLVVVGGVLACYSLLVTVAFIIFWVRSKRSRLLHSDY | |
| | MNMTPRRPGPTRKHYQPYAPPRDFAAYRSRVKFSRSADAP | |
| | AYQQGQNQLYNELNLGRREEYDVLDKRRGRDPEMGGKPRR | |
| | KNPQEGLYNELQKDKMAEAYSEIGMKGERRRGKGHDGLYQ | |
| | GLSTATKDTYDALHMQALPPR | |
| 15 | ATGGAGTGGTCCTGGGTGTTCCTGTTTTTCCTGAGCGTCAC | DNA sequence |
| | AACCGGTGTGCATAGTGATATTGAGCAGAAGCTGATTAGCG | encoding CD19 |
| | AAGAGGACCTGGATATCCAGATGACACAGACCACCAGCAGC | CAR2-63 |
| | CTGAGCGCCAGCCTGGGCGACCGAGTGACTATCAGCTGCCG | |
| | GGCATCCCAGGATATTTCTAAGTATCTGAACTGGTACCAGC | |
| | AGAAGCCCGACGGCACTGTCAAACTGCTGATCTACCACACC | |
| | AGTAGACTGCATTCAGGGGTGCCTAGCAGGTTCTCCGGATC | |
| | TGGCAGTGGGACTGACTACTCCCTGACCATCTCTAACCTGG | |
| | AGCAGGAAGATATTGCCACCTATTTCTGCCAGCAGGGCAAT | |
| | ACACTGCCTTACACTTTTGGCGGGGGAACAAAGCTGGAGAT | |
| | CACTGGCGGAGGAGGATCTGGAGGAGGAGGAAGTGGAGGAG | |
| | GAGGATCAGAGGTGAAACTGCAGGAAAGCGGACCAGGACTG | |
| | GTCGCACCTTCACAGAGCCTGTCCGTGACATGTACTGTCTC | |
| | CGGAGTGTCTCTGCCCGATTACGGCGTCTCTTGGATCCGGC | |
| | AGCCCCCTAGAAAGGGACTGGAGTGGCTGGGCGTGATCTGG | |
| | GGAAGTGAAACTACCTACTATAATAGTGCTCTGAAATCAAG | |
| | ACTGACCATCATTAAGGACAACTCTAAAAGTCAGGTGTTTC | |
| | TGAAGATGAATTCCCTGCAGACCGACGATACAGCAATCTAC | |
| | TATTGCGCCAAACACTACTATTACGGGGGAGCTATGCCATG | |
| | GATTACTGGGGGCAGGGAACTTCCGTCACCGTGAGCAGCgc | |
| | TAAGCCCACCACGACGCCAGCGCCGCGACCACCAACACCGG | |
| | CGCCCACCATCGCGTCGCAGCCCCTGTCCCTGCGCCCAGAG | |
| | GCGTGCCGGCCAGCGGCGGGGGGCGCAGIGCACACGAGGGG | |
| | GCTGGACTTCGCCCCTAGGAAAATTGAAGTTATGTATCCTC | |
| | CTCCTTACCTAGACAATGAGAAGAGCAATGGAACCATTATC | |
| | CATGTGAAAGGGAAACACCTTTGTCCAAGTCCCCTATTTCC | |
| | CGGACCTTCTAAGCCCTITTGGGIGCTGGIGGTGGTTGGTG | |
| | GAGTCCTGGCTTGCTATAGCTTGCTAGTAACAGTGGCCTTT | |
| | ATTATTTTCTGGGTGAGGAGTAAGAGGAGCAGGCTCCTGCA | |
| | CAGTGACTACATGAACATGACTCCCCGCCGCCCCGGGCCCA | |
| | CCCGCAAGCATTACCAGCCCTATGCCCCACCACGCGACTTC | |
| | GCAGCCTATCGCTCCAGAGTGAAGTTCAGCAGGAGCGCAGA | |
| | CGCCCCCGCGIACCAGCAGGGCCAGAACCAGCTCTATAACG | |
| | AGCTCAATCTAGGACGAAGAGAGGAGTACGATGITTIGGAC | |
| | AAGAGACGTGGCCGGGACCCTGAGATGGGGGGAAAGCCGAG | |
| | AAGGAAGAACCCTCAGGAAGGCCTGTACAATGAACTGCAGA | |
| | AAGATAAGATGGCGGAGGCCTACAGTGAGATTGGGATGAAA | |
| | GGCGAGCGCCGGAGGGGCAAGGGGCACGATGGCCTTTACCA | |
| | GGGTCTCAGTACAGCCACCAAGGACACCTACGACGCCCTTC | |
| | ACATGCAGGCCCTGCCCCCTCGCTAA | |
| 16 | aagcttgcatgcctgcaggtcgactctaggcacataaagaa | DNA sequence of the |
| | aaacataactaaccaagctgcagccgagacagtgaaaagaa | retroviral vector |
| | ccgttaaaacggtttgttttaaataaactgaattatttaga | encoding the CD19 |
| | gtcatttctttggtaggaaagtacattggcacgtaaaggag | CAR2-63 |
| | cccaaagcaatctgtggaaagcccaggctgggagcccagca | |
| | gtttgcatcccctcctggcgtgtacctaagggtttcttaat | |
| | tgtgtggtttctaaatcttccagagggtttgtctcattcac | |
| | ttccacttcggtgcacaatacttggacgcggatttactgtc | |
| | ttagcatctatcggtggcccttcgattgaggctgaacctga | |
| | ggcccacttcttcagcttgttaaggagagcacaagcaccag | |
| | aagaggctgacccggcagacctgtgggcatttttaacaagg | |
| | gcctcctgggtctgtgggaggcaggcttacataaggtgcaa | |
| | attagaaatataaataataagcccatatcaatttgtcatct | |
| | ttttttaagctcaagttttgaaagaccccacctgtaggttt | |
| | ggcaagctagcttaagtaacgccattttgcaaggcatggaa | |
| | aatacataactgagaatagagaagttcagatcaaggttagg | |
| | aacagagagacagcagaatatgggccaaacaggatatctgt | |
| | ggtaagcagttcctgccccgctcagggccaagaacagttgg | |
| | aacaggagaatatgggccaaacaggatatctgtggtaagca | |
| | gttcctgccccggctcagggccaagaacagatggtccccag | |
| | atgcggtcccgccctcagcagtttctagagaaccatcagat | |
| | gtttccagggtgccccaaggacctgaaatgaccctgtgcct | |
| | tatttgaactaaccaatcagttcgcttctcgcttctgttcg | |
| | cgcgcttctgctccccgagctcaataaaagagcccacaacc | |
| | cctcactcggcgcgccagtcctccgatagactgcgtcgccc | |
| | gggtacccgtattcccaataaagcctcttgctgtttgcatc | |
| | cgaatcgtggactcgctgatccttgggagggtctcctcaga | |
| | ttgattgactgcccacctcgggggtctttcatttggaggtt | |
| | ccaccgagatttggagacccctgcccagggaccaccgaccc | |
| | ccccgccgggaggtaagctggccagcaacttatctgtgtct | |
| | gtccgattgtctagtgtctatgactgattttatgcgcctgc | |
| | gtcggtactagttagctaactagctctgtatctggcggacc | |
| | cgtggtggaactgacgagttcggaacacccggccgcaaccc | |
| | tgggagacgtcccagggacttcgggggccgtttttgtggcc | |
| | cgacctgagtcctaaaatcccgatcgtttaggactctttgg | |
| | tgcaccccccttagaggagggatatgtggttctggtaggag | |
| | acgagaacctaaaacagttcccgcctccgtctgaatttttg | |
| | ctttcggtttgggaccgaagccgcgccgcgcgtcttgtctg | |
| | ctgcagcatcgttctgtgttgtctctgtctgactgtgtttc | |
| | tgtatttgtctgaaaatatgggcccgggctagactgttacc | |
| | actcccttaagtttgaccttaggtcactggaaagatgtcga | |
| | gcggatcgctcacaaccagtcggtagatgtcaagaagagac | |
| | gttgggttaccttctgctctgcagaatggccaacctttaac | |
| | gtcggatggccgcgagacggcacctttaaccgagacctcat | |
| | cacccaggttaagatcaaggtcttttcacctggcccgcatg | |
| | gacacccagaccaggtcccctacatcgtgacctgggaagcc | |
| | ttggcttttgacccccctccctgggtcaagccctttgtaca | |
| | ccctaagcctccgcctcctcttcctccatccgccccgtctc | |
| | tcccccttgaacctcctcgttcgaccccgcctcgatcctcc | |
| | ctttatccagccctcactccttctctaggcgcccccatatg | |
| | gccatatgagatcttatatggggcacccccgccccttgtaa | |
| | acttccctgaccctgacatgacaagagttactaacagcccc | |
| | tctctccaagctcacttacaggctctctacttagtccagca | |
| | cgaagtctggagacctctggcggcagcctaccaagaacaac | |
| | tggaccgaccggtggtacctcacccttaccgagtcggcgac | |
| | acagtgtgggtccgccgacaccagactaagaacctagaacc | |
| | tcgctggaaaggaccttacacagtcctgctgaccaccccca | |
| | ccgccctcaaagtagacggcatcgcagcttggatacacgcc | |
| | gcccacgtgaaggctgccgaccccgggggtggaccatcctc | |
| | tagactgcctcgcgaggatccctcgaggccgccaccATGGA | |
| | GTGGTCCTGGGTGTTCCTGTTTTTCCTGAGCGTCACAACCG | |
| | GTGTGCATAGTGATATTGAGCAGAAGCTGATTAGCGAAGAG | |
| | GACCTGGATATCCAGATGACACAGACCACCAGCAGCCTGAG | |
| | CGCCAGCCTGGGCGACCGAGTGACTATCAGCTGCCGGGCAT | |
| | CCCAGGATATTTCTAAGTATCTGAACTGGTACCAGCAGAAG | |
| | CCCGACGGCACTGTCAAACTGCTGATCTACCACACCAGTAG | |
| | ACTGCATTCAGGGGTGCCTAGCAGGTTCTCCGGATCTGGCA | |
| | GTGGGACTGACTACTCCCTGACCATCTCTAACCTGGAGCAG | |
| | GAAGATATTGCCACCTATTTCTGCCAGCAGGGCAATACACT | |
| | GCCTTACACTTTTGGCGGGGGAACAAAGCTGGAGATCACTG | |
| | GCGGAGGAGGATCTGGAGGAGGAGGAAGTGGAGGAGGAGGA | |
| | TCAGAGGTGAAACTGCAGGAAAGCGGACCAGGACTGGTCGC | |
| | ACCTTCACAGAGCCTGTCCGTGACATGTACTGTCTCCGGAG | |
| | TGTCTCTGCCCGATTACGGCGTCTCTTGGATCCGGCAGCCC | |
| | CCTAGAAAGGGACTGGAGTGGCTGGGCGTGATCTGGGGAAG | |
| | TGAAACTACCTACTATAATAGTGCTCTGAAATCAAGACTGA | |
| | CCATCATTAAGGACAACTCTAAAAGTCAGGTGTTTCTGAAG | |
| | ATGAATTCCCTGCAGACCGACGATACAGCAATCTACTATTG | |
| | CGCCAAACACTACTATTACGGGGGAGCTATGCCATGGATTA | |
| | CTGGGGGCAGGGAACTTCCGTCACCGTGAGCAGCgcTAAGC | |
| | CCACCACGACGCCAGCGCCGCGACCACCAACACCGGCGCCC | |
| | ACCATCGCGTCGCAGCCCCTGTCCCTGCGCCCAGAGGCGTG | |
| | CCGGCCAGCGGCGGGGGGCGCAGTGCACACGAGGGGGCTGG | |
| | ACTTCGCCCCTAGGAAAATTGAAGTTATGTATCCTCCTCCT | |
| | TACCTAGACAATGAGAAGAGCAATGGAACCATTATCCATGT | |
| | GAAAGGGAAACACCTTTGTCCAAGTCCCCTATTTCCCGGAC | |
| | CTTCTAAGCCCTTTTGGGTGCTGGTGGTGGTTGGTGGAGTC | |
| | CTGGCTTGCTATAGCTTGCTAGTAACAGTGGCCTTTATTAT | |
| | TTTCTGGGTGAGGAGTAAGAGGAGCAGGCTCCTGCACAGTG | |
| | ACTACATGAACATGACTCCCCGCCGCCCCGGGCCCACCCGC | |
| | AAGCATTACCAGCCCTATGCCCCACCACGCGACTTCGCAGC | |
| | CTATCGCTCCAGAGTGAAGTTCAGCAGGAGCGCAGACGCCC | |
| | CCGCGTACCAGCAGGGCCAGAACCAGCTCTATAACGAGCTC | |
| | AATCTAGGACGAAGAGAGGAGTACGATGTTTTGGACAAGAG | |
| | ACGTGGCCGGGACCCTGAGATGGGGGGAAAGCCGAGAAGGA | |
| | AGAACCCTCAGGAAGGCCTGTACAATGAACTGCAGAAAGAT | |
| | AAGATGGCGGAGGCCTACAGTGAGATTGGGATGAAAGGCGA | |
| | GCGCCGGAGGGGCAAGGGGCACGATGGCCTTTACCAGGGTC | |
| | TCAGTACAGCCACCAAGGACACCTACGACGCCCTTCACATG | |
| | CAGGCCCTGCCCCCTCGCTAAcagccaggggccgcccaagg | |
| | gcgaattcgcggccgcatcgatgcataggccttccggatta | |
| | gtccaatttgttaaagacaggatatcagtggtccaggctct | |
| | agttttgactcaacaatatcaccagctgaagcctatagagt | |
| | acgagccatagataaaataaaagattttatttagtctccag | |
| | aaaaaggggggaatgaaagaccccacctgtaggtttggcaa | |
| | gctagcttaagtaacgccattttgcaaggcatggaaaatac | |
| | ataactgagaatagagaagttcagatcaaggttaggaacag | |
| | agagacagcagaatatgggccaaacaggatatctgtggtaa | |
| | gcagttcctgccccgctcagggccaagaacagttggaacag | |
| | gagaatatgggccaaacaggatatctgtggtaagcagttcc | |
| | tgccccggctcagggccaagaacagatggtccccagatgcg | |
| | gtcccgccctcagcagtttctagagaaccatcagatgtttc | |
| | cagggtgccccaaggacctgaaatgaccctgtgccttattt | |
| | gaactaaccaatcagttcgcttctcgcttctgttcgcgcgc | |
| | ttctgctccccgagctcaataaaagagcccacaacccctca | |
| | ctcggcgcgccagtcctccgatagactgcgtcgcccgggta | |
| | cccgtgttctcaataaaccctcttgcagttgcatccgactc | |
| | gtggtctcgctgttccttgggagggtctcctctgagtgatt | |
| | gactacccgtcagcggggtctttcagtttctcccacctaca | |
| | caggtctcactaacattcctgatgtgccgcagggactccgt | |
| | cagcccggtttttgtttataataaaatgcaagaacagtgtt | |
| | cccttcaagccagactacatcctgactctcggctttataaa | |
| | agaatgttgaagggctctgtggactatctgccacacgactt | |
| | tttaagatttttatgcctcctggatgagggatttagtcaat | |
| | ctatcctcgtctattttgctggcttctccgtattttaaatt | |
| | tctagtttgcactcccttcctgagagcacggcgattgcaga | |
| | gtagttaatactctgagggcaggcttctgtgaaaaggttgc | |
| | ctgggctcagtgtgagattttgccataaaaaggggtcctgc | |
| | ccctgtgtacagacagatcggaatctagagtgcatactcag | |
| | agtccccgcggttccggggctctgatctcagggcatctttg | |
| | cctagagatcctctacgccggacgcatcgtggccgggtacc | |
| | gagctcgaattcgtaatcatggtcatagctgtttcctgtgt | |
| | gaaattgttatccgctcacaattccacacaacatacgagcc | |
| | ggaagcataaagtgtaaagcctggggtgcctaatgagtgag | |
| | ctaactcacattaattgcgttgcgctcactgcccgctttcc | |
| | agtcgggaaacctgtcgtgccagctgcattaatgaatcggc | |
| | caacgcgcggggagaggcggtttgcgtattgggcgctcttc | |
| | cgcttcctcgctcactgactcgctgcgctcggtcgttcggc | |
| | tgcggcgagcggtatcagctcactcaaaggcggtaatacgg | |
| | ttatccacagaatcaggggataacgcaggaaagaacatgtg | |
| | agcaaaaggccagcaaaaggccaggaaccgtaaaaaggccg | |
| | cgttgctggcgtttttccataggctccgcccccctgacgag | |
| | catcacaaaaatcgacgctcaagtcagaggtggcgaaaccc | |
| | gacaggactataaagataccaggcgtttccccctggaagct | |
| | ccctcgtgcgctctcctgttccgaccctgccgcttaccgga | |
| | tacctgtccgcctttctcccttcgggaagcgtggcgctttc | |
| | tcatagctcacgctgtaggtatctcagttcggtgtaggtcg | |
| | ttcgctccaagctgggctgtgtgcacgaaccccccgttcag | |
| | cccgaccgctgcgccttatccggtaactatcgtcttgagtc | |
| | caacccggtaagacacgacttatcgccactggcagcagcca | |
| | ctggtaacaggattagcagagcgaggtatgtaggcggtgct | |
| | acagagttcttgaagtggtggcctaactacggctacactag | |
| | aaggacagtatttggtatctgcgctctgctgaagccagtta | |
| | ccttcggaaaaagagttggtagctcttgatccggcaaacaa | |
| | accaccgctggtagcggtggtttttttgtttgcaagcagca | |
| | gattacgcgcagaaaaaaaggatctcaagaagatcctttga | |
| | tcttttctacggggtctgacgctcagtggaacgaaaactca | |
| | cgttaagggattttggtcatgagattatcaaaaaggatctt | |
| | cacctagatccttttaaattaaaaatgaagttttaaatcaa | |
| | tctaaagtatatatgagtaaacttggtctgacagttaccaa | |
| | tgcttaatcagtgaggcacctatctcagcgatctgtctatt | |
| | tcgttcatccatagttgcctgactccccgtcgtgtagataa | |
| | ctacgatacgggagggcttaccatctggccccagtgctgca | |
| | atgataccgcgagacccacgctcaccggctccagatttatc | |
| | agcaataaaccagccagccggaagggccgagcgcagaagtg | |
| | gtcctgcaactttatccgcctccatccagtctattaattgt | |
| | tgccgggaagctagagtaagtagttcgccagttaatagttt | |
| | gcgcaacgttgttgccattgctacaggcatcgtggtgtcac | |
| | gctcgtcgtttggtatggcttcattcagctccggttcccaa | |
| | cgatcaaggcgagttacatgatcccccatgttgtgcaaaaa | |
| | agcggttagctccttcggtcctccgatcgttgtcagaagta | |
| | agttggccgcagtgttatcactcatggttatggcagcactg | |
| | cataattctcttactgtcatgccatccgtaagatgcttttc | |
| | tgtgactggtgagtactcaaccaagtcattctgagaatagt | |
| | gtatgcggcgaccgagttgctcttgcccggcgtcaatacgg | |
| | gataataccgcgccacatagcagaactttaaaagtgctcat | |
| | cattggaaaacgttcttcggggcgaaaactctcaaggatct | |
| | taccgctgttgagatccagttcgatgtaacccactcgtgca | |
| | cccaactgatcttcagcatcttttactttcaccagcgtttc | |
| | tgggtgagcaaaaacaggaaggcaaaatgccgcaaaaaagg | |
| | gaataagggcgacacggaaatgttgaatactcatactcttc | |
| | ctttttcaatattattgaagcatttatcagggttattgtct | |
| | catgagcggatacatatttgaatgtatttagaaaaataaac | |
| | aaataggggttccgcgcacatttccccgaaaagtgccacct | |
| | gacgtctaagaaaccattattatcatgacattaacctataa | |
| | aaataggcgtatcacgaggccctttcgtctcgcgcgtttcg | |
| | gtgatgacggtgaaaacctctgacacatgcagctcccggag | |
| | acggtcacagcttgtctgtaagcggatgccgggagcagaca | |
| | agcccgtcagggcgcgtcagcgggtgttggcgggtgtcggg | |
| | gctggcttaactatgcggcatcagagcagattgtactgaga | |
| | gtgcaccatatgcggtgtgaaataccgcacagatgcgtaag | |
| | gagaaaataccgcatcaggcgccattcgccattcaggctgc | |
| | gcaactgttgggaagggcgatcggtgcgggcctcttcgcta | |
| | ttacgccagctggcgaaagggggatgtgctgcaaggcgatt | |
| | aagttgggtaacgccagggttttcccagtcacgacgttgta | |
| | aaacgacggccagtgcc | |
| 22 | EVKLQESGPGLVAPSQSLSVTCTVSGVSLPDYGVSWIRQPP | CD19 scFv |
| | RKGLEWLGVIWGSETTYYNSALKSRLTIIKDNSKSQVFLKM | V_{H}-(G₄S)₃-V_{L} |
| | NSLQTDDTAIYYCAKHYYYGGSYAMDYWGQGTSVTVSSGGG | |
| | GSGGGGSGGGGSDIQMTQTTSSLSASLGDRVTISCRASQDI | |
| | SKYLNWYQQKPDGTVKLLIYHTSRLHSGVPSRESGSGSGTD | |
| | YSLIISNLEQEDIATYFCQQGNTLPYTFGGGTKLEIT | |
| 23 | DIEQKLISEEDLDIQMTQTTSSLSASLGDRVTISCRASQDI | CD19 CAR2-63 |
| | SKYLNWYQQKPDGTVKLLIYHTSRLHSGVPSRFSGSGSGTD | (mature polypeptide, |
| | YSLTISNLEQEDIATYFCQQGNTLPYTFGGGTKLEITGGGG | no signal peptide) |
| | SGGGGSGGGGSEVKLQESGPGLVAPSQSLSVTCTVSGVSLP | |
| | DYGVSWIRQPPRKGLEWLGVIWGSETTYYNSALKSRLTIIK | |
| | DNSKSQVFLKMNSLQTDDTAIYYCAKHYYYGGSYAMDYWGQ | |
| | GTSVTVSSAKPTTTPAPRPPTPAPTIASQPLSLRPEACRPA | |
| | AGGAVHTRGLDFAPRKIEVMYPPPYLDNEKSNGTIIHVKGK | |
| | HLCPSPLFPGPSKPFWVLVVVGGVLACYSLLVTVAFIIFWV | |
| | RSKRSRLLHSDYMNMTPRRPGPTRKHYQPYAPPRDFAAYRS | |
| | RVKFSRSADAPAYQQGQNQLYNELNLGRREEYDVLDKRRGR | |
| | DPEMGGKPRRKNPQEGLYNELQKDKMAEAYSEIGMKGERRR | |
| | GKGHDGLYQGLSTATKDTYDALHMQALPPR | |
| 24 | MEWSWVFLFFLSVTTGVHSDIQMTQTTSSLSASLGDRVTIS | CD19 CAR2-63a |
| | CRASQDISKYLNWYQQKPDGTVKLLIYHTSRLHSGVPSRFS | (precursor |
| | GSGSGTDYSLTISNLEQEDIATYFCQQGNTLPYTFGGGTKL | with signal |
| | EITGGGGSGGGGSGGGGSEVKLQESGPGLVAPSQSLSVTCT | peptide) |
| | VSGVSLPDYGVSWIRQPPRKGLEWLGVIWGSETTYYNSALK | |
| | SRLTIIKDNSKSQVFLKMNSLQTDDTAIYYCAKHYYYGGSY | |
| | AMDYWGQGTSVTVSSAKPTITPAPRPPTPAPTIASQPLSLR | |
| | PEACRPAAGGAVHTRGLDFAPRKIEVMYPPPYLDNEKSNGT | |
| | IIHVKGKHLCPSPLFPGPSKPFWVLVVVGGVLACYSLLVTV | |
| | AFIIFWVRSKRSRLLHSDYMNMTPRRPGPTRKHYQPYAPPR | |
| | DFAAYRSRVKFSRSADAPAYQQGONOLYNELNLGRREEYDV | |
| | LDKRRGRDPEMGGKPRRKNPQEGLYNELQKDKMAEAYSEIG | |
| | MKGERRRGKGHDGLYQGLSTATKDTYDALHMQALPPR | |
| 25 | ATGGAATGGTCATGGGTCTTTCTCTTTTTTCTCAGCGTGAC | DNA sequence |
| | CACCGGAGTCCACTCCGATATCCAGATGACACAGACCACCA | encoding CD19 |
| | GCAGCCTGAGCGCCAGCCTGGGCGACCGAGTGACTATCAGC | CAR2-63a |
| | TGCCGGGCATCCCAGGATATTTCTAAGTATCTGAACTGGTA | |
| | CCAGCAGAAGCCCGACGGCACTGTCAAACTGCTGATCTACC | |
| | ACACCAGTAGACTGCATTCAGGGGTGCCTAGCAGGTTCTCC | |
| | GGATCTGGCAGTGGGACTGACTACTCCCTGACCATCTCTAA | |
| | CCTGGAGCAGGAAGATATTGCCACCTATTTCTGCCAGCAGG | |
| | GCAATACACTGCCTTACACTTTTGGCGGGGGAACAAAGCTG | |
| | GAGATCACTGGCGGAGGAGGATCTGGAGGAGGAGGAAGTGG | |
| | AGGAGGAGGATCAGAGGTGAAACTGCAGGAAAGCGGACCAG | |
| | GACTGGTCGCACCTTCACAGAGCCTGTCCGTGACATGTACT | |
| | GTCTCCGGAGTGTCTCTGCCCGATTACGGCGTCTCTTGGAT | |
| | CCGGCAGCCCCCTAGAAAGGGACTGGAGTGGCTGGGCGTGA | |
| | TCTGGGGAAGTGAAACTACCTACTATAATAGTGCTCTGAAA | |
| | TCAAGACTGACCATCATTAAGGACAACTCTAAAAGTCAGGT | |
| | GTTTCTGAAGATGAATTCCCTGCAGACCGACGATACAGCAA | |
| | TCTACTATTGCGCCAAACACTACTATTACGGCGGGAGCTAT | |
| | GCCATGGATTACTGGGGGCAGGGAACTTCCGTCACCGTGAG | |
| | CAGCgcTAAGCCCACCACGACGCCAGCGCCGCGACCACCAA | |
| | CACCGGCGCCCACCATCGCGTCGCAGCCCCTGTCCCTGCGC | |
| | CCAGAGGCGTGCCGGCCAGCGGCGGGGGGCGCAGTGCACAC | |
| | GAGGGGGCTGGACTTCGCCCCTAGGAAAATTGAAGTTATGT | |
| | ATCCTCCTCCTTACCTAGACAATGAGAAGAGCAATGGAACC | |
| | ATTATCCATGTGAAAGGGAAACACCTTTGTCCAAGTCCCCT | |
| | ATTTCCCGGACCTTCTAAGCCCTTTTGGGTGCTGGTGGTGG | |
| | TTGGTGGAGTCCTGGCTTGCTATAGCTTGCTAGTAACAGTG | |
| | GCCTTTATTATTTTCTGGGTGAGGAGTAAGAGGAGCAGGCT | |
| | CCTGCACAGTGACTACATGAACATGACTCCCCGCCGCCCCG | |
| | GGCCCACCCGCAAGCATTACCAGCCCTATGCCCCACCACGC | |
| | GACTTCGCAGCCTATCGCTCCAGAGTGAAGTTCAGCAGGAG | |
| | CGCAGACGCCCCCGCGTACCAGCAGGGCCAGAACCAGCTCT | |
| | ATAACGAGCTCAATCTAGGACGAAGAGAGGAGTACGATGTT | |
| | TTGGACAAGAGACGTGGCCGGGACCCTGAGATGGGGGGAAA | |
| | GCCGAGAAGGAAGAACCCTCAGGAAGGCCTGTACAATGAAC | |
| | TGCAGAAAGATAAGATGGCGGAGGCCTACAGTGAGATTGGG | |
| | ATGAAAGGCGAGCGCCGGAGGGGCAAGGGGCACGATGGCCT | |
| | TTACCAGGGTCTCAGTACAGCCACCAAGGACACCTACGACG | |
| | CCCTTCACATGCAGGCCCTGCCCCCTCGCTAA | |
| 26 | aagcttgcatgcctgcaggtcgactctaggcacataaagaa | DNA sequence of the |
| | aaacataactaaccaagctgcagccgagacagtgaaaagaa | retroviral vector |
| | ccgttaaaacggtttgttttaaataaactgaattatttaga | encoding the CD19 |
| | gtcatttctttggtaggaaagtacattggcacgtaaaggag | CAR2-63a (from start |
| | cccaaagcaatctgtggaaagcccaggctgggagcccagca | codon to stop codon) |
| | gtttgcatcccctcctggcgtgtacctaagggtttcttaat | |
| | tgtgtggtttctaaatcttccagagggtttgtctcattcac | |
| | ttccacttcggtgcacaatacttggacgcggatttactgtc | |
| | ttagcatctatcggtggcccttcgattgaggctgaacctga | |
| | ggcccacttcttcagcttgttaaggagagcacaagcaccag | |
| | aagaggctgacccggcagacctgtgggcatttttaacaagg | |
| | gcctcctgggtctgtgggaggcaggcttacataaggtgcaa | |
| | attagaaatataaataataagcccatatcaatttgtcatct | |
| | ttttttaagctcaagttttgaaagaccccacctgtaggttt | |
| | ggcaagctagcttaagtaacgccattttgcaaggcatggaa | |
| | aatacataactgagaatagagaagttcagatcaaggttagg | |
| | aacagagagacagcagaatatgggccaaacaggatatctgt | |
| | ggtaagcagttcctgccccgctcagggccaagaacagttgg | |
| | aacaggagaatatgggccaaacaggatatctgtggtaagca | |
| | gttcctgccccggctcagggccaagaacagatggtccccag | |
| | atgcggtcccgccctcagcagtttctagagaaccatcagat | |
| | gtttccagggtgccccaaggacctgaaatgaccctgtgcct | |
| | tatttgaactaaccaatcagttcgcttctcgcttctgttcg | |
| | cgcgcttctgctccccgagctcaataaaagagcccacaacc | |
| | cctcactcggcgcgccagtcctccgatagactgcgtcgccc | |
| | gggtacccgtattcccaataaagcctcttgctgtttgcatc | |
| | cgaatcgtggactcgctgatccttgggagggtctcctcaga | |
| | ttgattgactgcccacctcgggggtctttcatttggaggtt | |
| | ccaccgagatttggagacccctgcccagggaccaccgaccc | |
| | ccccgccgggaggtaagctggccagcaacttatctgtgtct | |
| | gtccgattgtctagtgtctatgactgattttatgcgcctgc | |
| | gtcggtactagttagctaactagctctgtatctggcggacc | |
| | cgtggtggaactgacgagttcggaacacccggccgcaaccc | |
| | tgggagacgtcccagggacttcgggggccgtttttgtggcc | |
| | cgacctgagtcctaaaatcccgatcgtttaggactctttgg | |
| | tgcaccccccttagaggagggatatgtggttctggtaggag | |
| | acgagaacctaaaacagttcccgcctccgtctgaatttttg | |
| | ctttcggtttgggaccgaagccgcgccgcgcgtcttgtctg | |
| | ctgcagcatcgttctgtgttgtctctgtctgactgtgtttc | |
| | tgtatttgtctgaaaatatgggcccgggctagactgttacc | |
| | actcccttaagtttgaccttaggtcactggaaagatgtcga | |
| | gcggatcgctcacaaccagtcggtagatgtcaagaagagac | |
| | gttgggttaccttctgctctgcagaatggccaacctttaac | |
| | gtcggatggccgcgagacggcacctttaaccgagacctcat | |
| | cacccaggttaagatcaaggtcttttcacctggcccgcatg | |
| | gacacccagaccaggtcccctacatcgtgacctgggaagcc | |
| | ttggcttttgacccccctccctgggtcaagccctttgtaca | |
| | ccctaagcctccgcctcctcttcctccatccgccccgtctc | |
| | tcccccttgaacctcctcgttcgaccccgcctcgatcctcc | |
| | ctttatccagccctcactccttctctaggcgcccccatatg | |
| | gccatatgagatcttatatggggcacccccgccccttgtaa | |
| | acttccctgaccctgacatgacaagagttactaacagcccc | |
| | tctctccaagctcacttacaggctctctacttagtccagca | |
| | cgaagtctggagacctctggcggcagcctaccaagaacaac | |
| | tggaccgaccggtggtacctcacccttaccgagtcggcgac | |
| | acagtgtgggtccgccgacaccagactaagaacctagaacc | |
| | tcgctggaaaggaccttacacagtcctgctgaccaccccca | |
| | ccgccctcaaagtagacggcatcgcagcttggatacacgcc | |
| | gcccacgtgaaggctgccgaccccgggggtggaccatcctc | |
| | tagactgcctcgcgaggatccCTCGAGGCCGCCACCATGGA | |
| | ATGGTCATGGGTCTTTCTCTTTTTTCTCAGCGTGACCACCG | |
| | GAGTCCACTCCGATATCCAGATGACACAGACCACCAGCAGC | |
| | CTGAGCGCCAGCCTGGGCGACCGAGTGACTATCAGCTGCCG | |
| | GGCATCCCAGGATATTTCTAAGTATCTGAACTGGTACCAGC | |
| | AGAAGCCCGACGGCACTGTCAAACTGCTGATCTACCACACC | |
| | AGTAGACTGCATTCAGGGGTGCCTAGCAGGTTCTCCGGATC | |
| | TGGCAGTGGGACTGACTACTCCCTGACCATCTCTAACCTGG | |
| | AGCAGGAAGATATTGCCACCTATTTCTGCCAGCAGGGCAAT | |
| | ACACTGCCTTACACTTTTGGCGGGGGAACAAAGCTGGAGAT | |
| | CACTGGCGGAGGAGGATCTGGAGGAGGAGGAAGTGGAGGAG | |
| | GAGGATCAGAGGTGAAACTGCAGGAAAGCGGACCAGGACTG | |
| | GTCGCACCTTCACAGAGCCTGTCCGTGACATGTACTGTCTC | |
| | CGGAGTGTCTCTGCCCGATTACGGCGTCTCTTGGATCCGGC | |
| | AGCCCCCTAGAAAGGGACTGGAGTGGCTGGGCGTGATCTGG | |
| | GGAAGTGAAACTACCTACTATAATAGTGCTCTGAAATCAAG | |
| | ACTGACCATCATTAAGGACAACTCTAAAAGTCAGGTGTTTC | |
| | TGAAGATGAATTCCCTGCAGACCGACGATACAGCAATCTAC | |
| | TATTGCGCCAAACACTACTATTACGGCGGGAGCTATGCCAT | |
| | GGATTACTGGGGGCAGGGAACTTCCGTCACCGTGAGCAGCg | |
| | cTAAGCCCACCACGACGCCAGCGCCGCGACCACCAACACCG | |
| | GCGCCCACCATCGCGTCGCAGCCCCTGTCCCTGCGCCCAGA | |
| | GGCGTGCCGGCCAGCGGGGGGGGCGCAGTGCACACGAGGGG | |
| | GCTGGACTTCGCCCCTAGGAAAATTGAAGTTATGTATCCTC | |
| | CTCCTTACCTAGACAATGAGAAGAGCAATGGAACCATTATC | |
| | CATGTGAAAGGGAAACACCTTTGTCCAAGTCCCCTATTTCC | |
| | CGGACCTTCTAAGCCCTTTTGGGTGCTGGTGGTGGTTGGTG | |
| | GAGTCCTGGCTTGCTATAGCTTGCTAGTAACAGTGGCCTTT | |
| | ATTATTTTCTGGGTGAGGAGTAAGAGGAGCAGGCTCCTGCA | |
| | CAGTGACTACATGAACATGACTCCCCGCCGCCCCGGGCCCA | |
| | CCCGCAAGCATTACCAGCCCTATGCCCCACCACGCGACTTC | |
| | GCAGCCTATCGCTCCAGAGTGAAGTTCAGCAGGAGCGCAGA | |
| | CGCCCCCGCGTACCAGCAGGGCCAGAACCAGCTCTATAACG | |
| | AGCTCAATCTAGGACGAAGAGAGGAGTACGATGTTTTGGAC | |
| | AAGAGACGTGGCCGGGACCCTGAGATGGGGGGAAAGCCGAG | |
| | AAGGAAGAACCCTCAGGAAGGCCTGTACAATGAACTGCAGA | |
| | AAGATAAGATGGCGGAGGCCTACAGTGAGATTGGGATGAAA | |
| | GGCGAGCGCCGGAGGGGCAAGGGGCACGATGGCCTTTACCA | |
| | GGGTCTCAGTACAGCCACCAAGGACACCTACGACGCCCTTC | |
| | ACATGCAGGCCCTGCCCCCTCGCTAACAGCCAGGGGCCGCC | |
| | CAAGGGCGAATTCGCggccgcatcgatcataggccttccgg | |
| | attagtccaatttgttaaagacaggatatcagtggtccagg | |
| | ctctagttttgactcaacaatatcaccagctgaagcctata | |
| | gagtacgagccatagataaaataaaagattttatttagtct | |
| | ccagaaaaaggggggaatgaaagaccccacctgtaggtttg | |
| | gcaagctagcttaagtaacgccattttgcaaggcatggaaa | |
| | atacataactgagaatagagaagttcagatcaaggttagga | |
| | acagagagacagcagaatatgggccaaacaggatatctgtg | |
| | gtaagcagttcctgccccgctcagggccaagaacagttgga | |
| | acaggagaatatgggccaaacaggatatctgtggtaagcag | |
| | ttcctgccccggctcagggccaagaacagatggtccccaga | |
| | tgcggtcccgccctcagcagtttctagagaaccatcagatg | |
| | tttccagggtgccccaaggacctgaaatgaccctgtgcctt | |
| | atttgaactaaccaatcagttcgcttctcgcttctgttcgc | |
| | gcgcttctgctccccgagctcaataaaagagcccacaaccc | |
| | ctcactcggcgcgccagtcctccgatagactgcgtcgcccg | |
| | ggtacccgtgttctcaataaaccctcttgcagttgcatccg | |
| | actcgtggtctcgctgttccttgggagggtctcctctgagt | |
| | gattgactacccgtcagcggggtctttcagtttctcccacc | |
| | tacacaggtctcactaacattcctgatgtgccgcagggact | |
| | ccgtcagcccggtttttgtttataataaaatgcaagaacag | |
| | tgttcccttcaagccagactacatcctgactctcggcttta | |
| | taaaagaatgttgaagggctctgtggactatctgccacacg | |
| | actttttaagatttttatgcctcctggatgagggatttagt | |
| | caatctatcctcgtctattttgctggcttctccgtatttta | |
| | aatttctagtttgcactcccttcctgagagcacggcgattg | |
| | cagagtagttaatactctgagggcaggcttctgtgaaaagg | |
| | ttgcctgggctcagtgtgagattttgccataaaaaggggtc | |
| | ctgcccctgtgtacagacagatcggaatctagagtgcatac | |
| | tcagagtccccgcggttccggggctctgatctcagggcatc | |
| | tttgcctagagatcctctacgccggacgcatcgtggccggg | |
| | taccgagctcgaattcgtaatcatggtcatagctgtttcct | |
| | gtgtgaaattgttatccgctcacaattccacacaacatacg | |
| | agccggaagcataaagtgtaaagcctggggtgcctaatgag | |
| | tgagctaactcacattaattgcgttgcgctcactgcccgct | |
| | ttccagtcgggaaacctgtcgtgccagctgcattaatgaat | |
| | cggccaacgcgcggggagaggcggtttgcgtattgggcgct | |
| | cttccgcttcctcgctcactgactcgctgcgctcggtcgtt | |
| | cggctgcggcgagcggtatcagctcactcaaaggcggtaat | |
| | acggttatccacagaatcaggggataacgcaggaaagaaca | |
| | tgtgagcaaaaggccagcaaaaggccaggaaccgtaaaaag | |
| | gccgcgttgctggcgtttttccataggctccgcccccctga | |
| | cgagcatcacaaaaatcgacgctcaagtcagaggtggcgaa | |
| | acccgacaggactataaagataccaggcgtttccccctgga | |
| | agctccctcgtgcgctctcctgttccgaccctgccgcttac | |
| | cggatacctgtccgcctttctcccttcgggaagcgtggcgc | |
| | tttctcatagctcacgctgtaggtatctcagttcggtgtag | |
| | gtcgttcgctccaagctgggctgtgtgcacgaaccccccgt | |
| | tcagcccgaccgctgcgccttatccggtaactatcgtcttg | |
| | agtccaacccggtaagacacgacttatcgccactggcagca | |
| | gccactggtaacaggattagcagagcgaggtatgtaggcgg | |
| | tgctacagagttcttgaagtggtggcctaactacggctaca | |
| | ctagaaggacagtatttggtatctgcgctctgctgaagcca | |
| | gttaccttcggaaaaagagttggtagctcttgatccggcaa | |
| | acaaaccaccgctggtagcggtggtttttttgtttgcaagc | |
| | agcagattacgcgcagaaaaaaaggatctcaagaagatcct | |
| | ttgatcttttctacggggtctgacgctcagtggaacgaaaa | |
| | ctcacgttaagggattttggtcatgagattatcaaaaagga | |
| | tcttcacctagatccttttaaattaaaaatgaagttttaaa | |
| | tcaatctaaagtatatatgagtaaacttggtctgacagtta | |
| | ccaatgcttaatcagtgaggcacctatctcagcgatctgtc | |
| | tatttcgttcatccatagttgcctgactccccgtcgtgtag | |
| | ataactacgatacgggagggcttaccatctggccccagtgc | |
| | tgcaatgataccgcgagacccacgctcaccggctccagatt | |
| | tatcagcaataaaccagccagccggaagggccgagcgcaga | |
| | agtggtcctgcaactttatccgcctccatccagtctattaa | |
| | ttgttgccgggaagctagagtaagtagttcgccagttaata | |
| | gtttgcgcaacgttgttgccattgctacaggcatcgtggtg | |
| | tcacgctcgtcgtttggtatggcttcattcagctccggttc | |
| | ccaacgatcaaggcgagttacatgatcccccatgttgtgca | |
| | aaaaagcggttagctccttcggtcctccgatcgttgtcaga | |
| | agtaagttggccgcagtgttatcactcatggttatggcagc | |
| | actgcataattctcttactgtcatgccatccgtaagatgct | |
| | tttctgtgactggtgagtactcaaccaagtcattctgagaa | |
| | tagtgtatgcggcgaccgagttgctcttgcccggcgtcaat | |
| | acgggataataccgcgccacatagcagaactttaaaagtgc | |
| | tcatcattggaaaacgttcttcggggcgaaaactctcaagg | |
| | atcttaccgctgttgagatccagttcgatgtaacccactcg | |
| | tgcacccaactgatcttcagcatcttttactttcaccagcg | |
| | tttctgggtgagcaaaaacaggaaggcaaaatgccgcaaaa | |
| | aagggaataagggcgacacggaaatgttgaatactcatact | |
| | cttcctttttcaatattattgaagcatttatcagggttatt | |
| | gtctcatgagcggatacatatttgaatgtatttagaaaaat | |
| | aaacaaataggggttccgcgcacatttccccgaaaagtgcc | |
| | acctgacgtctaagaaaccattattatcatgacattaacct | |
| | ataaaaataggcgtatcacgaggccctttcgtctcgcgcgt | |
| | ttcggtgatgacggtgaaaacctctgacacatgcagctccc | |
| | ggagacggtcacagcttgtctgtaagcggatgccgggagca | |
| | gacaagcccgtcagggcgcgtcagcgggtgttggcgggtgt | |
| | cggggctggcttaactatgcggcatcagagcagattgtact | |
| | gagagtgcaccatatgcggtgtgaaataccgcacagatgcg | |
| | taaggagaaaataccgcatcaggcgccattcgccattcagg | |
| | ctgcgcaactgttgggaagggcgatcggtgcgggcctcttc | |
| | gctattacgccagctggcgaaagggggatgtgctgcaaggc | |
| | gattaagttgggtaacgccagggttttcccagtcacgacgt | |
| | tgtaaaacgacggccagtgcc | |
| 27 | DIQMTQTTSSLSASLGDRVTISCRASQDISKYLNWYQQKPD | CD19 CAR2-63a |
| | GTVKLLIYHTSRLHSGVPSRFSGSGSGTDYSLTISNLEQED | (mature polypeptide, |
| | IATYFCQQGNTLPYTFGGGTKLEITGGGGSGGGGSGGGGSE | no signal peptide) |
| | VKLQESGPGLVAPSQSLSVTCTVSGVSLPDYGVSWIRQPPR | |
| | KGLEWLGVIWGSETTYYNSALKSRLTIIKDNSKSQVFLKMN | |
| | SLQTDDTAIYYCAKHYYYGGSYAMDYWGQGISVTVSSAKPT | |
| | TTPAPRPPTPAPTIASQPLSLRPEACRPAAGGAVHTRGLDF | |
| | APRKIEVMYPPPYLDNEKSNGTIIHVKGKHLCPSPLFPGPS | |
| | KPFWVLVVVGGVLACYSLLVTVAFIIFWVRSKRSRLLHSDY | |
| | MNMTPRRPGPTRKHYQPYAPPRDFAAYRSRVKFSRSADAPA | |
| | YQQGQNQLYNELNLGRREEYDVLDKRRGRDPEMGGKPRRKN | |
| | PQEGLYNELQKDKMAEAYSEIGMKGERRRGKGHDGLYQGLS | |
| | TATKDTYDALHMQALPPR | |

### Example 2: Transduction of T Cells Isolated From PBMCs and Cord Blood With AntiCD19 CAR Construct

A gammaretroviral vector encoding the CD19 CAR2-63 CAR of SEQ ID NO:14 flanked by 5′ and 3′ retroviral LTRs (FIG. 2; SEQ ID NO:16) was introduced into T cells by retroviral transduction after activating the cells for 48-72 hours with an antibody to human CD3.

Activated human T cells isolated from either PBMCs or cord blood were prepared by thawing previously frozen normal healthy donor peripheral blood mononuclear cells (PBMC) or cord blood (CB) cells in a 37° C. water bath and then diluting the thawed cells into 10 ml AIM-V growth medium (GIBCO-Thermo Fisher scientific, Waltham, MA) supplemented with 10% FBS and 300 u/ml IL-2. The cells were transferred to a T25 culture flask to which 50 ng/ml mouse anti-human CD3 antibody OKT3 (Orth Biotech, Rartian, NJ) was added. The cells were incubated for two days at 37° C.

Frozen retroviral supernatant produced from PG13 packaging cells that had been transfected with the retroviral vector that included the CD19 CAR construct (SEQ ID NO:16) was thawed in a 37° C. water bath. The activated PBMC or cord blood activated T cells were centrifuged (1500 rpm for 5 min) and resuspended in the retroviral supernatant of PG13 packaging cells (1.5 ml/well, 2 wells total) to which IL-2 (1000 u/ml) was added, then transferred to the wells of 12-well plates (2 wells at 2×10⁶ cells/well) that had been coated with retronectin (10 μg/ml in PBS). The plates were parafilmed and centrifuged at 2,500 rpm at room temperature for 1 hour and then incubated for a further 3 hours at 37° C. The transduction process was repeated (the plate was centrifuged, the supernatant was removed and the cells were resuspended in fresh packaging cell viral supernatant, and centrifuged for 1 hour at room temperature). The twice-transduced cells were incubated for 1 hour at 37° C., after which an additional 1 ml of media supplemented with FBS and CD3 antibody was added to each well and the cells were incubated for 3 to 4 days at 37° C.

### Example 3: Flow Cytometric Analysis of CD19 CAR-Transduced T Cells

For detecting the expression of CD19 on the cell surface of transduced T cells isolated from PBMCs and CB, flow cytometric assays were performed. For detection of the T cell receptor, 10⁶ cells were centrifuged at 1500 rpm for 5 min, then the cells were resuspended in 50 μl CD19-Fc protein to a concentration of 8 μg/ml. The cells plus CD19 protein were incubated for 1 hour at room temperature, after with 1 ml of PBS was added as a wash and the cells were centrifuged at 1500 rpm for 5 min and resuspended in APC anti-human IgG Fcγ at 2 μg/ml. The cells were washed with 1 ml PBS (centrifuged at 1500 rpm for 5 min) and resuspended in FACS staining buffer. The samples were analyzed on an Intellicyte flow cytometer.

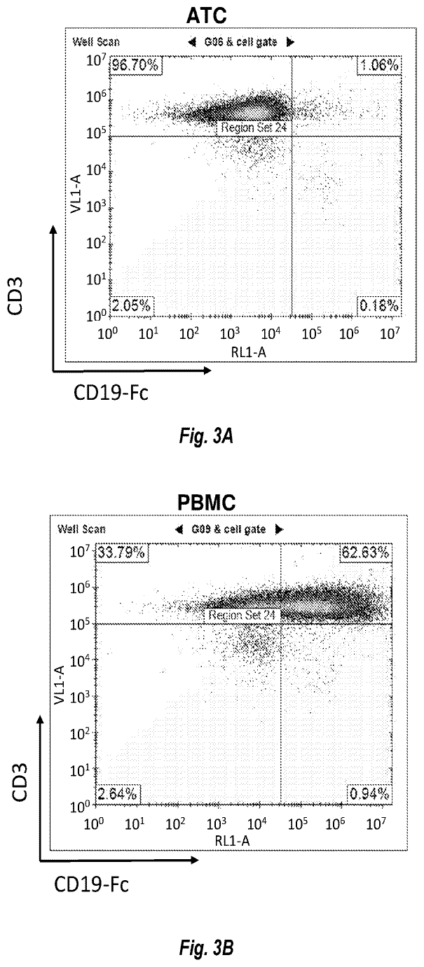

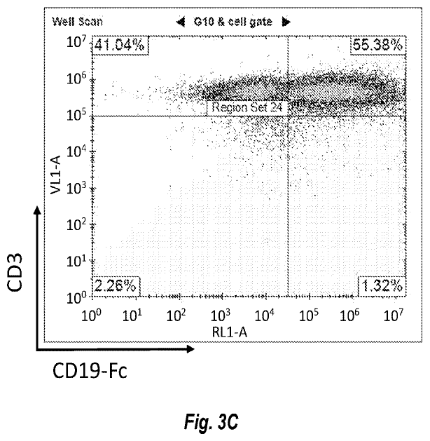

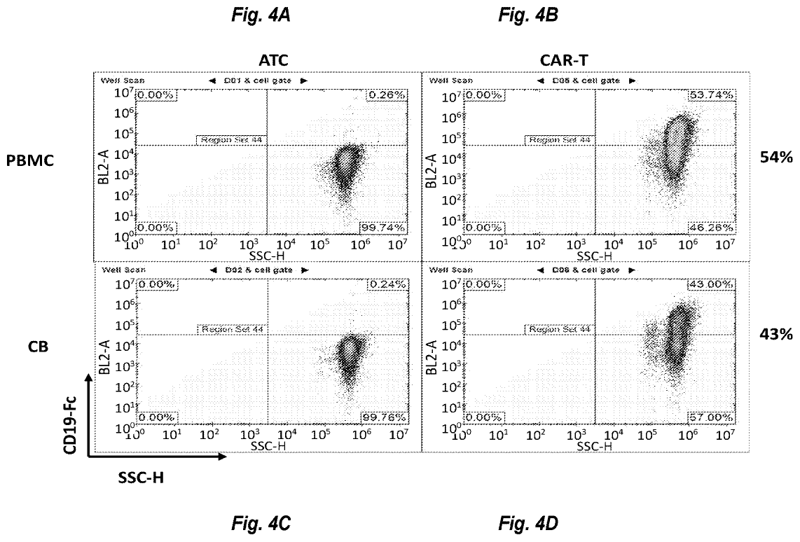

FIGS. 3A-C show the flow cytometry results from a first transduction experiment, in which activated T cells from both PBMCs and CB cells were transduced with the CD19 CAR2-63 retrovirus. Activated T cells that were not transduced with the CD19 CAR2-63 retrovirus did not express the CD19 CAR (FIG. 3A). Approximately 62.6% of CD19 CAR2-63-transduced PBMC-derived T cells expressed the anti-CD19 antibody (FIG. 3B), and 55.4% of CB-derived T cells (FIG. 3C) were positive for the anti-CD19 antibody. FIGS. 4A-D show the flow cytometry results from a second independent transduction experiment in which activated T cells from both PBMCs and CB cells were transduced with the CD19 CAR2-63 retrovirus. In this experiment, nontransduced activated T cells isolated from PBMCs (FIG. 4A) or cord blood (FIG. 4C) did not express the CD19 CAR, whereas 54% of CD19 CAR2-63 transduced PBMC-derived T cells (FIG. 4B) and 43% of CD19 CAR2-63 transduced CB-derived T cells (FIG. 4C) expressed the CD19 CAR.

### Example 4: Activation Assays of CD19 CAR-Transduced T Cells

Non-transduced control T cells from CB and PBMCs and antiCD19 CAR2-63-transduced T cells from CB and PBMCs were separately stimulated in microtiter plates with K562 cells, K562 cells transfected with a CD19 gene, or Raji tumor cells which normally express CD19. For the cytokine production assay, 1×10⁵ cells/well non-transduced control T cells or CAR2-antiCD19 CAR-transduced T cells were mixed with 1×10⁵ cells/well K562 cells, K562 cells expressing CD19, or Raji tumor cells in growth medium. After culturing for 24 hours, culture supernatants were harvested and measured for IFNγ, IL-2, and TNFα with ELISA assay kits from eBioscience (San Diego, CA). All experiments were done in triplicate.

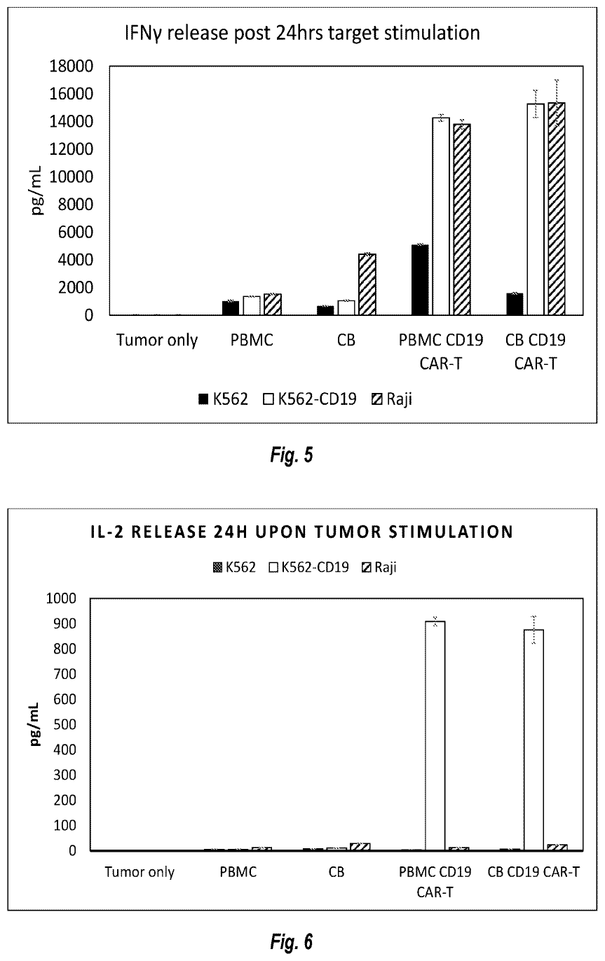

FIG. 5 shows that non-transduced T cells isolated from PBMCs released less than 2000 pg/ml IFNγ when cultured with K562 cells, regardless of whether the K562 cells expressed CD19. CB-derived non-transduced T cells produced a modest amount of IFNγ only when co-cultured with Raji cells. In contrast, both PBMC-derived and CB-derived T cells that were transduced with the construct encoding CD19 CAR2-63 dramatically increased production of IFNγ when co-cultured with CD19-expressing cells (either K562 cells expressing a CD19 transgene or Raji cells). Transduced PBMC-derived T cells produced more than 8-fold the amount of IFNγ produced by non-transduced PBMC-derived T cells when co-cultured with CD19+ cells. CB-derived T cells transduced with the CD19 CAR produced more than three-fold the IFNγ produced by non-transduced CB T cells when co-cultured with non-CD19-expressing cells. Both types of T-cell (PBMC-derived and cord blood-derived) produced much more IFNγ when co-cultured with CD19+ cells—CB-derived cells than when co-cultured with CD19− cells. For example, CD19 CAR2-63 transduced CB T cells produced at least 2.5 times as much IFNγ when co-cultured with CD19+cells, and CD19 CAR2-63 transduced PBMC T cells produced nearly 10 times as much IFNγ when co-cultured with CD19+ cells.

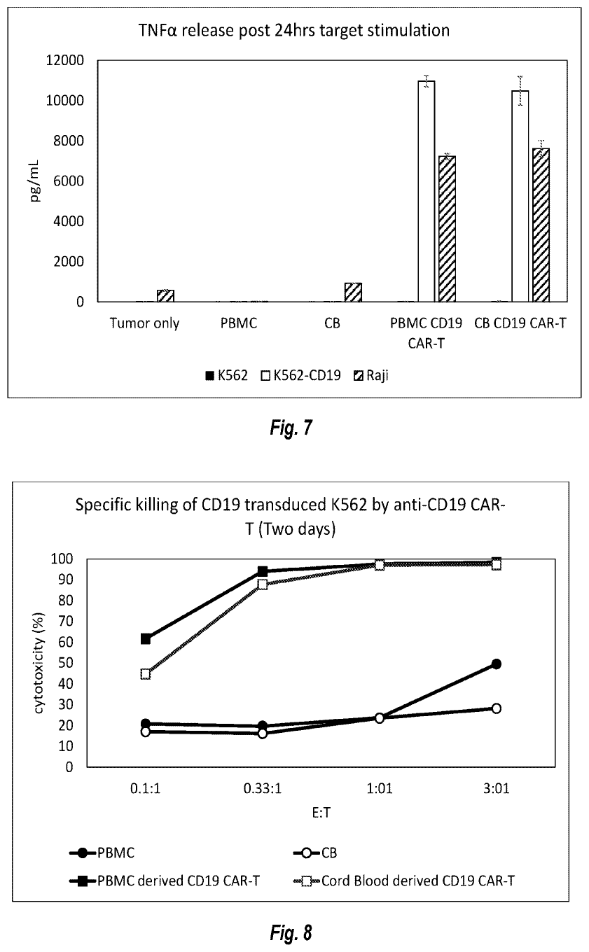

FIG. 6 shows that production of IL-2 by T cells derived from either PBMCs or CB that expressed the CD19 CAR2-63 (but not non-transduced T cells of either source) released a large amount of IL-2 when co-cultured with CD19-expressing K562 cells. FIG. 7 shows that TNFα release by CD19 CAR2-63-expressing CB and PBMC T cells occurred when they were co-cultured with CD-19-expressing K562 cells and (CD19+) Raji cells, but not when co-cultured with K562 cells that did not express CD19. Non-CAR transduced CB and PBMC T cells produced very little TNFα, and only when co-cultured with Raji cells.

### Example 5: Cytotoxicity Assay Using CD19 CAR-Transduced T Cells

Cytotoxicity of CAR2-AntiCD19 CAR-T cells was also assessed. One assay for measuring cytotoxicity is the DELFIA cytotoxicity assay (PerkinElmer, Waltham, MA). In this assay, target cells (e.g., tumor cells) were first loaded with fluorescence enhancing ligand for 25 minutes at 37° C. 2.5×10³ cells/well of K562 cells or 5×10³ cells/well. Raji tumor cells were then mixed with non-transduced control or transduced T cells at different effector:target (E:T) ratios and incubated for 2 hours. 20 μl/well supernatant was harvested and analyzed for the released ligand by adding europium solution to form a fluorescent chelate. Time-Resolved Fluorescence (TRF) can be measured on a TRF capable plate reader Cytation 5 (BioTek instruments, Winooski, VT). Cytotoxicity can be calculated by using the following formula: % Specific Lysis=(Experimental−Spontancous)/(Maximum−Spontancous)*100.

FIG. 8 shows that when cytotoxicity assays were performed using PBMC or CB derived CD19 CAR-expressing T cells as the effector cells against CD19-expressing K562 cells, % cytotoxicity levels increased steadily as the effector:target ratio increased from 0.1:1 to 0.33:1, and peaked at 97% at a 1:1 effector:target ratio. When the effector cells were non-transduced CB-derived or PBMC-derived T cells, PBMC-derived T cells demonstrated a higher % cytotoxicity than was reached by CB-derived T cells, but the % cytotoxicity only reached 49.5% at a 3:1 3:1 effector:target ratio (and 28% for CB-derived T cells as effectors).

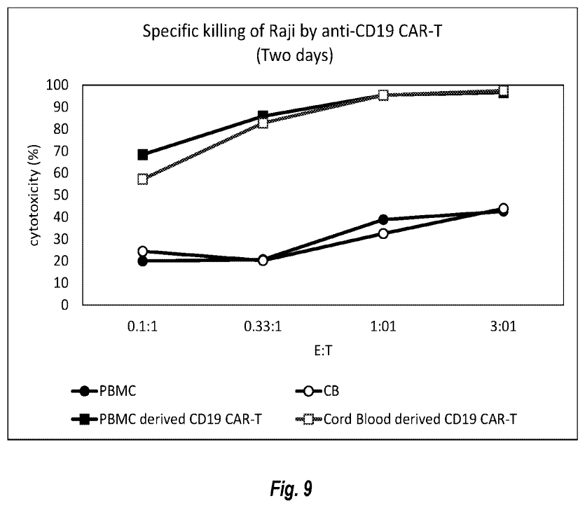

FIG. 9 shows that when cytotoxicity assays are performed using PBMC or CB derived CD19 CAR-expressing T cells as the effector cells against Raji cells which naturally express CD19, % cytotoxicity levels increase steadily as the effector:target ratio increases from 0.1:1 to 1:1, at which point the cytotoxicity begins to level off at greater than 95%. When the effector cells are non-transduced CB-derived or PBMC-derived T cells however, cytotoxicity only reaches about 39% at the 1:1 ratio, and only climbs slightly higher, to about 44%, at the 3:1 ratio.

### Example 6. Isolation of gdT Cells

For some experiments, gamma delta T cells (gdT cells) were isolated from peripheral blood mononuclear cells (PBMCs). Gamma delta T cell isolation can be performed using the Stemcell Technologies Human Gamma/Delta T Cell Isolation Kit (Stemcell Technologies, Seattle, WA).

In one protocol, freshly thawed PBMC suspensions are suspended in 30 mL Dulbecco's Phosphate Buffered Saline (DPBS) containing 25% fetal bovine serum (FBS). (PBMCs are isolated from Leukopaks ordered through HemaCare and frozen at a concentration of 1×10⁸ cells per ml in vials. For gdT cell isolation, ten vials can be thawed into 30 mL DPBS medium containing 25% FBS in a single 50 mL centrifuge tube, resulting in approximately 8×10⁸-1×10⁹ cells per 50 mL tube.) The PBMCs are passed through a 40 μm cell strainer and cell number was determined. Approximately 3×10⁵ cells can be reserved for flow cytometry and the remainder are harvested by centrifugation. The supernatant is removed and the cell pellet is respuspended in 60 μL MACS separation buffer (Miltenyi Biotech, San Diego, CA) per 10⁷ cells in a 50 mL tube, to which 20 μL FcR blocking reagent per 10⁷ cells is added. The cells are incubated with blocking reagent for 5 minutes at room temperature, after which 12.5 L of EasySep™ Human Gamma/Delta T Cell Isolation Cocktail (Stemcell Technologies, Seattle, WA) is added per 5×10⁷ cells. After mixing briefly, the cells are incubated a further 15 min at room temperature with mixing on a plate shaker. The cells are then washed to remove unbound primary antibody by adding 1-2 mL of buffer per 10⁷ cells and centrifuging at 1400 rpm for 5 min. The supernatant is aspirated and the cell pellet is resuspended in MACS separation buffer (80 μL per 10⁷ cells).

For pan cell depletion, magnetic particles (anti-biotin microbeads, Miltenyi Biotech) are vortexed before removing 12.5 μL of suspended beads per 5×10⁷ cells and adding them to the suspended cell preparation. The cells and magnetic beads are incubated for 10 min without shaking at room temperature, and then additional MACS separation buffer is added to bring the volume up to 25 mL (if the original volume was less than 10 mL) or 50 mL (if the original volume was greater than 10 mL). The cells are pipeted up and down gently 2-3 times to mix and the tube (without lid) is placed into the magnet stand (MACS Column Separator, Miltenyi Biotec) for 10 min at RT. The enriched cell suspension is carefully pipeted into a new 50 mL tube. The cells are centrifuged at 1400 rpm for 5 min, after which the supernatant is removed. The cells are then resuspended at approximately 10⁷ cells/mL in MACS separation buffer.

2.5 μL anti-TCR α/β-biotin human antibodies (Miltenyi Biotec) are added per 10⁷ cells to the pan cell depleted cells and the antibodies and cells are mixed with pipette tips and then incubated for 10 min at 4° C. in the dark. The cells are then washed by adding 13 mL MACS separation buffer, transferring the suspended cells to a 15 mL tube, centrifuging at 1400 rpm for 5 min, and aspirating the supernatant. The wash is repeated and the final pellet is resuspended in 97.5 μL MACS separation buffer per 10⁷ cells and 2.5 ul/10⁷cells anti-Biotin Microbeads are then added to the cells. The suspension is pipeted a few times to mix, mixed with pipet tips, and incubated 15 min in the dark at 4° C.

The cells are then washed by adding 13 mL buffer and centrifuging the sample at 1400 rpm for 5 min. The supernatant is aspirated completely and the cells are resuspended in up to 500 uL MACS separation buffer/10⁸cells.

For depletion of α/β cells, the LD column (Miltenyi Biotec) is rinsed with 2 mL of MACS separation buffer and the cell suspension is applied to the column. The flow through of unlabeled cells is collected and the column is washed 5 times with 1 mL of buffer each time. The washes are added to the flow through and the cell number is determined. An aliquot of 3×10⁵ cells is removed for flow cytometry to assess cell purity. The remaining cells are spun down and resuspended in T cell medium to a concentration of 2×10⁶ cells per mL and dispensed into wells of a 6 well culture plate.

For expansion of T cells, T cell TransAct solution (Miltenyi Biotec, 5 μL per 10⁶ cells) is added to the cells in T Cell Medium, for example T cell OpTmizer™ CTS™ Medium (Fisher Scientific) modified to include 1% glutamax, 5% human serum, 26 ml of OpTmizer™ T-Cell Expansion Supplement, 1:1000 gentamicin, and 300U IL-2. The plate is placed in a cell incubator for 2-3 days. The culture medium is then exchanged with fresh T cell medium without added T cell TransAct solution (Miltenyi Biotec).

On day 9, during the exponential phase of T cell growth, the gdT cells are transferred to a suitable tissue culture bag. The cells are maintained at a density of 0.5×10⁶ cells/ml. On day 13, the cells are counted after resuspension and transferred to a tissue culture bag to keep the cell density at 0.5×10⁶ cells/ml. On day 16, the cells are again counted and thereafter the cell density is maintained at 1×10⁶ cells/ml in culture medium containing 300 U/ml rIL-2 in a tissue culture bag. On day 20, the cells are counted and frozen.

### Example 7: Treatment of Raji Xenografts in NSG Mice

For in vivo studies, T cells were isolated from PBMCs essentially as described and transduced with viral supernatant from cultured PG13 packaging cells that had been transfected with the construct encoding the CD19 CAR2-63 (SEQ ID NO: 14) depicted in FIG. 2 (SEQ ID NO:16) essentially as described in Example 2 except that cells were activated with CD3 antibody (OKT3 at 50 ng/ml) for three days prior to transduction, and transduction was performed by centrifuging a six well plate that included T cells plus viral supernatant in the wells for one hour at 37° C., after which the plate was incubated for 1 hour at 37° C., and then 3 additional ml of culture media was added and the plate was incubated overnight. The transduction process was repeated the next day and the cells were then incubated for three days at 37° C. in tissue culture flasks before analyzing by flow cytometry.

In addition, in some experiments isolated gamma delta T cells (gdT cells) were transduced with the CD19 CAR2-63 construct for use in in vivo experiments. The gd T cells were isolated from PBMCs using pan cell depletion with negative selection kit and αβ T cell depletion with αβ antibodies. The purity and cell number of gamma delta T cells were assessed by FACS.

Isolated γδ T cells were activated with soluble CD3/CD28 antibody from MACS for 72 hrs, and then transduced with retroviral supernatant produced from PG13 cells that had been packaged with the CD19 CAR2-63-encoding retroviral vector. The transduction process was performed twice in succession. The transduction efficiency was examined 4 days after transduction by flow cytometry. The percentage of total CAR+T cells was calculated. Pan ATC (non-transduced), Pan T CD19 CAR2-63, gdT cells ATC (non-transduced), and CD19 CAR2-63 gdT cells were expanded in culture to reach the total cell number for further in vitro and in vivo studies. The transduction efficiency was examined by flow cytometry prior to freezing the cells for further studies.

TABLE 2
Experiment Groups

| | | | Treatment at Day 0 | |
|---|---|---|---|---|
| | | Group | (one day after tumor injection) | |
| Group | Treatment | size | Treatment amount | TE |
| 1 | untreated Control | 10 | n/a | n/a |
| 2 | ATC | 10 | matched total cell number | n/a |
| 3 | CAR-T | 10 | 1 × 10⁷ CAR+ cells | 43% |
| 4 | γδ-T | 10 | matched total cell number | n/a |
| 5 | γδ-CAR-T | 10 | 1 × 10⁷ CAR+ cells | 40% |
| 6 | CAR-T | 5 | 2 × 10⁶ CAR+ cells | 43% |
| 7 | γδ-CAR-T | 5 | 2 × 10⁶ CAR+ cells | 40% |

All animal experiments were treated and maintained in compliance with guidelines for care and use of laboratory animals. Eight-week-old female NSG immunodeficient mice purchased from The Jackson Laboratory (Bar Harbor, ME) were inoculated intravenously (i.v.) on Day −1 with 1×10⁶ Luc-GFP labelled Raji cells via tail veil. One day later (Day 0), T cells according to the table below were administered intravenously.

Animals were monitored closely for body weight loss, hind limb paralysis, loss of mobility, or signs of distress. Animals experiencing hind limb paralysis, moribund behavior, or body weight loss of greater than 20% were euthanized.

Tumor burden was measured weekly by bioluminescent imaging using an IVIS Spectrum In Vivo Imaging System (PerkinElmer, Waltham, MA) weekly. Peripheral blood samples were taken by tail vein bleeding at 3 hours, day 1, day 2, then weekly after T cell injection and were pooled together by groups. T cell engraftment and expansion was measured by flow cytometric assays with APC-conjugated mouse anti-human CD3 and PE-conjugated mouse anti-myc antibodies. Blood levels of human cytokine IFNγ, IL2, and TNFα were measured by using Bio-Rad Luminex Assays (Bio-Rad, Hercules, CA). Animals were monitored for signs of disease progression and overt toxicity, such as graft versus host disease (GVHD), as evidenced by >15% loss in body weight, loss of fur, and becoming moribund. The endpoint for the survival study was the day when the mice lost more than 15% of body weight or became moribund.

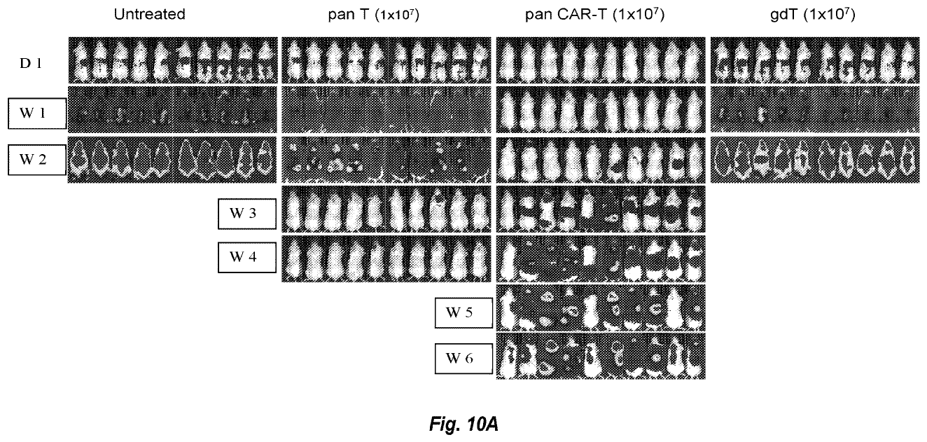

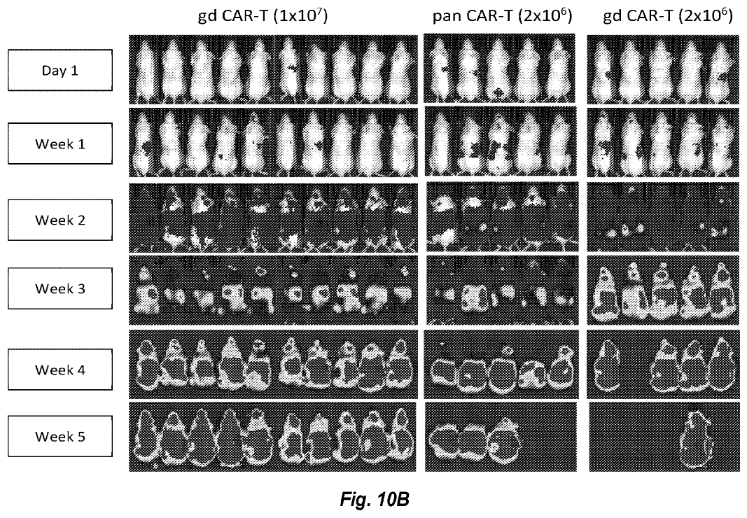

Images of the mice over the course of the six week study are provided in FIGS. 10A and B. FIG. 10A shows that untreated mice and mice treated with unmodified γδ T cells did not survive beyond Week 2, while mice treated with unmodified pan T cells (ATCs) did not survive beyond Week 4, succumbing to Graft-versus-Host Disease. Pan T cells expressing the CD19 CAR construct (Group 3) had the best survival, with all 10 mice surviving at 6 weeks. FIG. 10B shows that all 10 Group 5 mice treated with 10⁷ CD19 CAR-expressing γδ T cells survived to Week 5. Groups 6 and 7, treated with fewer CAR-T cells, had fewer mice surviving to Week 6: only 3 of 10 mice of Group 6 treated with 2×10⁶ (pan) CAR-T cells and 1 of 5 mice of Group 7 treated with 2×10⁶ γδ CAR-T cells survived to Week 6.

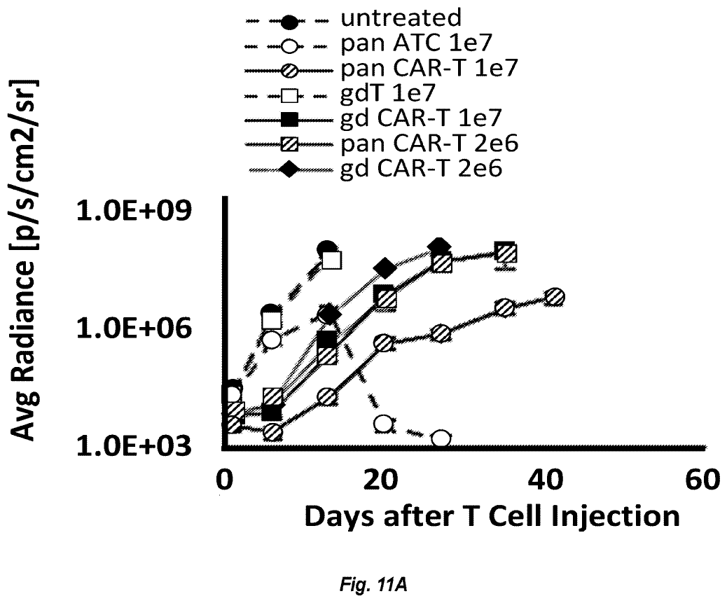

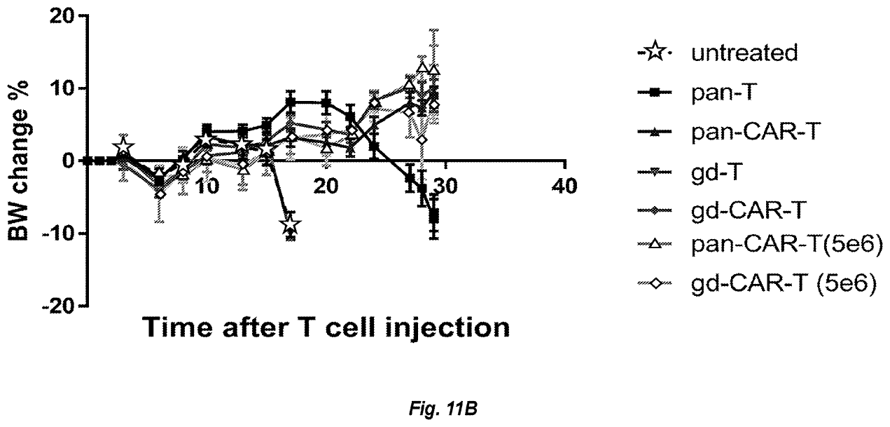

FIG. 11A provides a graph of tumor sizes based on irradiance in the treatment groups. Tumors grew most rapidly in the groups that were untreated or treated with unmodified γδ T cells (γδ T cells not transduced with the CD19 CAR construct). Mice treated with 1×10⁷ of the pan T cells (not transduced with the CAR construct) had tumors that initially grew rapidly but then shrank. Tumors grew after a brief lag in mice treated with 2×10⁶ γδ CAR-T cells, 1 x 10⁷ γδ CAR-T cells, and 2×10⁶ pan T cells (not transduced with the CAR construct). Tumors grew most slowly in mice treated with 1×10⁷ CAR-T cells. FIG. 11B provides the body weights of the mice in each group over the same time period, showing the drastic weight loss in the untreated, pan-T, and gdT treated groups.

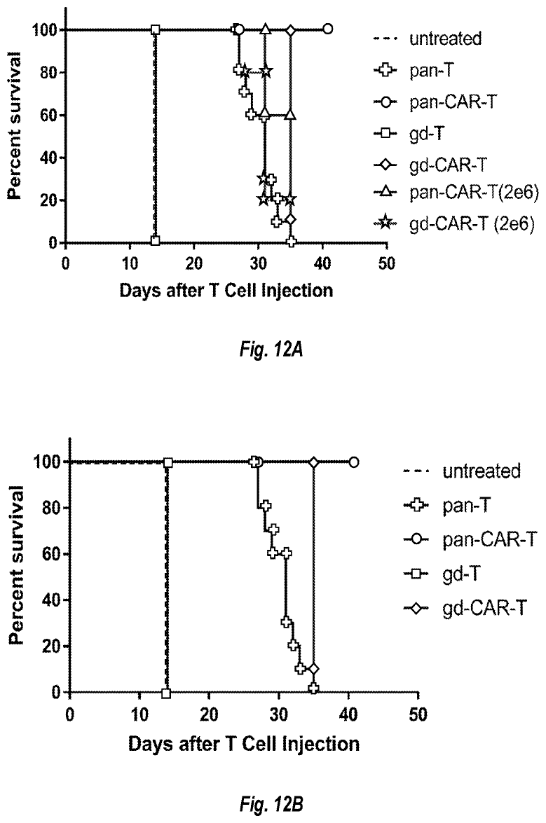

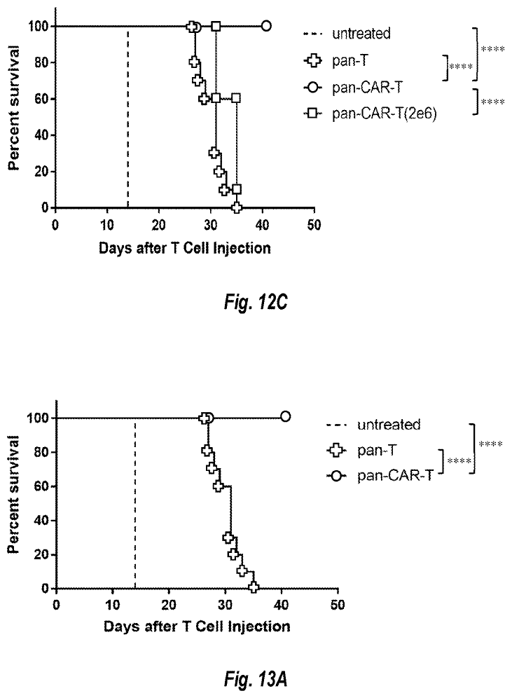

FIG. 12A provides survivorship curves of the treatment groups. The drop off in survivorship at fourteen days of the mice that did not receive any cell treatment (dashed line) and mice that received unmodified γδ T cells (squares) is evident. The greatest survivorship was demonstrated in the mice treated with pan T CAR-T cells (circles): none of the mice of this treatment group died by the end of the six week study. Mice treated with gdT CAR-T cells survived for five weeks. FIG. 12B provides a further graph of survivorship where Groups 6 and 7 receiving the lower cell doses of CAR-T cells have been removed. The pan T treatment group (no CAR expression) shows a decline in survivorship beginning at day 28 that resulted in all mice succumbing by day 35. The gd CAR-T treatment group shows survival until day 35 at which time all mice succumb (vertical line). The horizontal line demonstrates that all mice of the pan CAR-T cell treatment group survived through day 42. FIG. 12C provides a further graph of survivorship where Groups 4, 5, and 7 treated with gdT cells have been removed. The graph compares survivorship of untreated mice (vertical line at day 14), mice treated with pan T cells (decline in survivorship between day 28 and day 35), mice treated with pan CART-T cells (horizontal line showing no mortality), and mice treated with a lower dose of pan CART-T cells (all mice succumb by day 35).

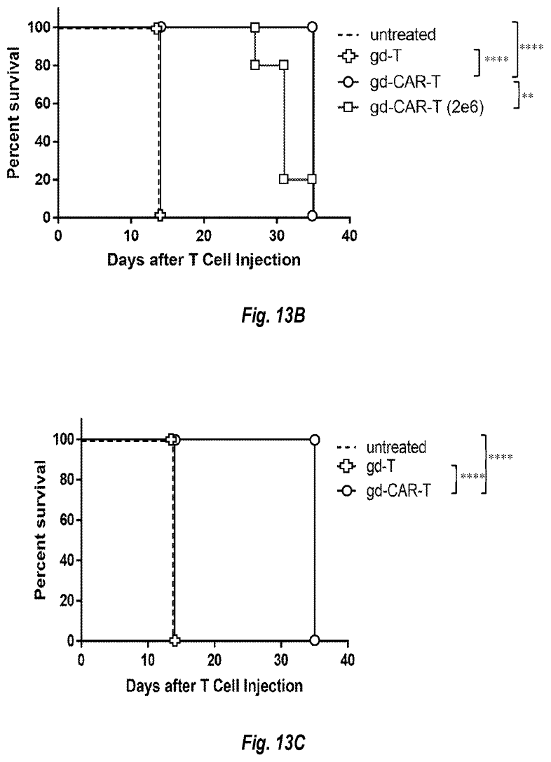

FIG. 13A provides a further graph providing the survivorship for the untreated group (vertical line at day 14), pan T cell treatment group (survivorship declining from day 28 to day 35), and the pan-CAR-T cell treatment group (horizontal line showing no decline in survivorship throughout the 6 week experiment). FIG. 13B compares survivorship for the untreated group (vertical line at day 14), gdT cell treatment group (survivorship to day 14), gdT 10⁷ cell treatment group (survivorship to day 35), and the gdT 2×10⁶ cell treatment group (survivorship declining from day 28 through 35). FIG. 13C compares survivorship for the untreated group (vertical line at day 14), gdT cell treatment group (survivorship to day 14), and the gdT 10⁷ cell treatment group (survivorship to day 35.

The significance level for differences in survivorship of some of the groups is provided in Table 3.

TABLE 3
Survivorship Significance Levels

| | | | | | | |
|---|---|---|---|---|---|---|
| | | | | | pan | gd |
| | | pan | | gd | CAR-T | CAR-T |
| untreated | pan T | CAR-T | gdT | CAR-T | 2e6 | 2e6 |

| | | | | |
|---|---|---|---|---|
| untreated | >0.0001 **** | >0.0001 **** | | |
| pan T | >0.0001 **** | | | |
| pan CAR-T | | >0.0001 **** | >0.0001 **** | |
| gdT | | >0.0001 **** | | |
| gdCAR-T | | | | 0.0011 |
| pan CAR-T | | | | 0.17 (ns) |
| 2e6 | | | | |
| gd CAR-T | | | | |
| 2e6 | | | | |

1. A CD19 chimeric antigen receptor (CAR) nucleic acid construct comprising a nucleic acid sequence encoding:
i) an scFv antibody that binds to CD19, wherein the scFv antibody comprises a heavy chain variable (VH) domain comprising an amino acid sequence that is at least 95% identical to the amino acid sequence of SEQ ID NO: 1 and a light chain variable (VL) domain comprising an amino acid sequence that is at least 95% identical to the amino acid sequence of SEQ ID NO:2;
ii) a hinge region of at least 65 amino acids in length,;
iii) a transmembrane domain; and
iv) a first intracellular domain having at least 95% identity to SEQ ID NO:7 and a second intracellular domain having at least 95% identity to SEQ ID NO:8;
wherein a T cell expressing the CAR encoded by the construct has exhibits higher cytotoxicity toward CD19-positive tumor cells than is exhibited by T cells not expressing the CAR encoded by the construct.

2. The CD19 CAR construct of claim 1, wherein the scFv antibody comprises a peptide linker between the heavy chain variable (VH) domain and the light chain variable (VL) domain, or wherein the peptide linker comprises the amino acid sequence of SEQ ID NO:3.

3. (canceled)

4. The CD19 CAR construct of claim 1, wherein the scFv antibody comprises the amino acid sequence of SEQ ID NO:12 or an amino acid sequence having at least 95% identity to SEQ ID NO:12.

5. The CD19 CAR construct of claim 1, wherein the hinge region comprises a first amino acid sequence having at least 95% identity to SEQ ID NO:4 and a second amino acid sequence having at least 95% identity to SEQ ID NO:5.

6. The CD19 CAR construct of claim 1, wherein the hinge region comprises an amino acid sequence having at least 95% identity to SEQ ID NO:13.

7. The CD19 CAR construct of claim 1, wherein the transmembrane region comprises an amino acid sequence having at least 95% identity to SEQ ID NO:6.

8. The CD19 CAR construct of claim 1, wherein the CAR comprises a signal peptide, and/or wherein the CAR comprises a myc tag (SEQ ID NO:15).

9. (canceled)

10. The CD19 CAR construct of claim 1, wherein the CAR comprises an amino acid sequence that is at least 95% identical to the amino acid sequence of SEQ ID NO:24, or wherein the CAR comprises the amino acid sequence of SEQ ID NO:24.

11. (canceled)

12. The CD19 CAR construct of claim 1, wherein the CAR comprises an amino acid sequence that is at least 95% identical to the amino acid sequence of SEQ ID NO:14.

13. A nucleic acid molecule comprising a CD19 CAR construct of claim 1, wherein the CAR construct is operably linked to a promoter.

14. A vector comprising a CAR construct of claim 1, optionally further comprising a promoter operably linked to the CAR construct.

15. (canceled)

16. The vector of claim 14, wherein the vector is a lentiviral vector, er a retroviral vector, or a gammaretroviral vector, optionally wherein the lentiviral vector, the retroviral vector, or the gammaretroviral vector comprises a promoter operably linked to the CAR construct.

17. (canceled)

18. (canceled)

19. A host cell, or a population of host cells, which express a CD19 CAR according to claim 1.

20. The host cell or population of host cells of claim 19, which are selected from the group consisting of a PBMC-derived T cell (or population thereof), a placental derived T cell (or a population thereof), and a cord blood derived T cell (or population thereof).

21. The host cell or population of host cells of claim 20, wherein the host cell or cells comprise a retroviral or lentiviral expression vector that encodes the CD19 CAR.

22. (canceled)

23. The host cell or population of host cells of claim 19, wherein the host cell or cells are pan T cells or gd T cells, optionally wherein the host cell or cells comprise a retroviral expression vector that encodes the CD19 CAR.

24. (canceled)

25. A method of treating a cancer in a subject, comprising administering to the subject a population of host cells according to claim 19.

26. A method of inhibiting growth of a tumor expressing CD19 in a patient, comprising administering to the patient a population of T cells comprising a CD19 CAR according to claim 19.

27. The method according to claim 26, wherein the population of T cells are isolated from PBMCs or wherein the population of T cells are isolated from placental tissue or cord blood, optionally wherein the population of T cells are allogeneic with respect to the subject or patient.

28. (canceled)

29. (canceled)

30. The method according to claim 25, wherein the cancer is a hematological cancer, optionally wherein the cancer is non-Hodgkin's lymphoma (NHL), B chronic lymphocytic leukemia (B-CLL), or B acute lymphocytic leukemia (ALL).

31. (canceled)


SEQUENCE LISTING (27 sequences)

SEQ ID NO: 1 - protein, 107 aa, Artificial Sequence
  Anti-CD19 antibody, variable light chain
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 2 - protein, 120 aa, Artificial Sequence
  Anti-CD19 antibody,  variable heavy chain
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 15 aa, Artificial Sequence
  peptide linker connecting VH and VL- amino acid
GGGGSGGGGSGGGGS

SEQ ID NO: 4 - protein, 46 aa, Artificial Sequence
  CD8 hinge region- amino acid
AKPTTTPAPRPPTPAPTIASQPLSLRPEACRPAAGGAVHTRGLDFA

SEQ ID NO: 5 - protein, 40 aa, Artificial Sequence
  CD28 extracellular domain/ spacer- amino acid
KIEVMYPPPYLDNEKSNGTIIHVKGKHLCPSPLFPGPSKP

SEQ ID NO: 6 - protein, 27 aa, Artificial Sequence
  CD28 transmembrane domain- amino acid
FWVLVVVGGVLACYSLLVTVAFIIFWV

SEQ ID NO: 7 - protein, 41 aa, Artificial Sequence
  CD28 intracellular signaling domain- amino acid
RSKRSRLLHSDYMNMTPRRPGPTRKHYQPYAPPRDFAAYRS

SEQ ID NO: 8 - protein, 113 aa, Artificial Sequence
  CD3-zeta signaling domain- amino acid
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 9 - protein, 19 aa, Artificial Sequence
  signal peptide from mouse antibody heavy chain - amino acid
MEWSWVFLFFLSVTTGVHS

SEQ ID NO: 10 - protein, 10 aa, Artificial Sequence
  myc tag - amino acid
EQKLISEEDL

SEQ ID NO: 11 - protein, 12 aa, Artificial Sequence
  myc tag with 2 additional amino acids - amino acid
DIEQKLISEEDL

SEQ ID NO: 12 - protein, 242 aa, Artificial Sequence
  CD19 scFv - amino acid
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 13 - protein, 88 aa, Artificial Sequence
  Hinge sequence- amino acid
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 14 - protein, 541 aa, Artificial Sequence
  CD19 CAR2-63 - amino acid
MEWSWVFLFFLSVTTGVHSDIEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 1626 bp, Artificial Sequence
  DNA sequence encoding anti-CD19 CAR2-63
atggagtggtcctgggtgttcctgtttttcctgagcgtcacaaccggtgtgcatagtgatattgagcagaagctgattagcgaagaggacctggatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 16 - DNA, 8013 bp, Artificial Sequence
  DNA sequence of the retroviral vector encoding the anti-CD19 CAR2-63 (from start codon to stop codon)
aagcttgcatgcctgcaggtcgactctaggcacataaagaaaaacataactaaccaagctgcagccgagacagtgaaaagaaccgttaaaacggtttgttttaaataaactgaattatttagagtcatttctttggtaggaaagtacattggcacgtaaaggagcccaaagcaatctgtggaaagcccaggctgggagcccagcagtttgcatcccctcctggcgtgtacctaagggtttcttaattgtgtggtttctaaatcttccagagggtttgtctcattcacttccacttcggtgcacaatacttggacgcggatttactgtcttagcatctatcggtggcccttcgattgaggctgaacctgaggcccacttcttcagcttgttaaggagagcacaagcaccagaagaggctgacccggcagacctgtgggcatttttaacaagggcctcctgggtctgtgggaggcaggcttacataaggtgcaaattagaaatataaataataagcccatatcaatttgtcatctttttttaagctcaagttttgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgcctcgcgaggatccctcgaggccgccaccatggagtggtcctgggtgttcctgtttttcctgagcgtcacaaccggtgtgcatagtgatattgagcagaagctgattagcgaagaggacctggatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccaggggccgcccaagggcgaattcgcggccgcatcgatgcataggccttccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcggggtctttcagtttctcccacctacacaggtctcactaacattcctgatgtgccgcagggactccgtcagcccggtttttgtttataataaaatgcaagaacagtgttcccttcaagccagactacatcctgactctcggctttataaaagaatgttgaagggctctgtggactatctgccacacgactttttaagatttttatgcctcctggatgagggatttagtcaatctatcctcgtctattttgctggcttctccgtattttaaatttctagtttgcactcccttcctgagagcacggcgattgcagagtagttaatactctgagggcaggcttctgtgaaaaggttgcctgggctcagtgtgagattttgccataaaaaggggtcctgcccctgtgtacagacagatcggaatctagagtgcatactcagagtccccgcggttccggggctctgatctcagggcatctttgcctagagatcctctacgccggacgcatcgtggccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 17 - protein, 10 aa, Artificial Sequence
  IgG1 upper hinge sequence
EPKSCDKTHT

SEQ ID NO: 18 - protein, 9 aa, Artificial Sequence
  lower hinge/CH2 sequence
PAPELLGGP

SEQ ID NO: 19 - protein, 12 aa, Artificial Sequence
  CH2 region
SVFLFPPKPKDT

SEQ ID NO: 20 - protein, 23 aa, Artificial Sequence
  Hinge sequence
EPKSCDKTHTCPPCPAPELLGGP

SEQ ID NO: 21 - protein, 4 aa, Artificial Sequence
  Hinge sequence
  MISC_FEATURE (3)..(3): Xaa = Pro, Arg, or Ser
CPXC

SEQ ID NO: 22 - protein, 242 aa, Artificial Sequence
  CD19 scFv VH-(G4S)3-VL
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 23 - protein, 522 aa, Artificial Sequence
  CD19 CAR2-63 (mature polypeptide)
DIEQKLISEEDLDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 529 aa, Artificial Sequence
  CD19 CAR2-63a (precursor with signal peptide)
MEWSWVFLFFLSVTTGVHSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - DNA, 1590 bp, Artificial Sequence
  DNA sequence encoding CD19 CAR2-63a
atggaatggtcatgggtctttctcttttttctcagcgtgaccaccggagtccactccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 26 - DNA, 7976 bp, Artificial Sequence
  DNA sequence of the retroviral vector encoding the CD19 CAR2-63a
aagcttgcatgcctgcaggtcgactctaggcacataaagaaaaacataactaaccaagctgcagccgagacagtgaaaagaaccgttaaaacggtttgttttaaataaactgaattatttagagtcatttctttggtaggaaagtacattggcacgtaaaggagcccaaagcaatctgtggaaagcccaggctgggagcccagcagtttgcatcccctcctggcgtgtacctaagggtttcttaattgtgtggtttctaaatcttccagagggtttgtctcattcacttccacttcggtgcacaatacttggacgcggatttactgtcttagcatctatcggtggcccttcgattgaggctgaacctgaggcccacttcttcagcttgttaaggagagcacaagcaccagaagaggctgacccggcagacctgtgggcatttttaacaagggcctcctgggtctgtgggaggcaggcttacataaggtgcaaattagaaatataaataataagcccatatcaatttgtcatctttttttaagctcaagttttgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcccagggaccaccgacccccccgccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgcctcgcgaggatccctcgaggccgccaccatggaatggtcatgggtctttctcttttttctcagcgtgaccaccggagtccactccgatatccagatgacacagaccaccagcagcctgagcgccagcctgggcgaccgagtgactatcagctgccgggcatcccaggatatttctaagtatctgaactggtaccagcagaagcccgacggcactgtcaaactgctgatctaccacaccagtagactgcattcaggggtgcctagcaggttctccggatctggcagtgggactgactactccctgaccatctctaacctggagcaggaagatattgccacctatttctgccagcagggcaatacactgccttacacttttggcgggggaacaaagctggagatcactggcggaggaggatctggaggaggaggaagtggaggaggaggatcagaggtgaaactgcaggaaagcggaccaggactggtcgcaccttcacagagcctgtccgtgacatgtactgtctccggagtgtctctgcccgattacggcgtctcttggatccggcagccccctagaaagggactggagtggctgggcgtgatctggggaagtgaaactacctactataatagtgctctgaaatcaagactgaccatcattaaggacaactctaaaagtcaggtgtttctgaagatgaattccctgcagaccgacgatacagcaatctactattgcgccaaacactactattacggcgggagctatgccatggattactgggggcagggaacttccgtcaccgtgagcagcgctaagcccaccacgacgccagcgccgcgaccaccaacaccggcgcccaccatcgcgtcgcagcccctgtccctgcgcccagaggcgtgccggccagcggcggggggcgcagtgcacacgagggggctggacttcgcccctaggaaaattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaacagccaggggccgcccaagggcgaattcgcggccgcatcgatcataggccttccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccgctcagggccaagaacagttggaacaggagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgttctcaataaaccctcttgcagttgcatccgactcgtggtctcgctgttccttgggagggtctcctctgagtgattgactacccgtcagcggggtctttcagtttctcccacctacacaggtctcactaacattcctgatgtgccgcagggactccgtcagcccggtttttgtttataataaaatgcaagaacagtgttcccttcaagccagactacatcctgactctcggctttataaaagaatgttgaagggctctgtggactatctgccacacgactttttaagatttttatgcctcctggatgagggatttagtcaatctatcctcgtctattttgctggcttctccgtattttaaatttctagtttgcactcccttcctgagagcacggcgattgcagagtagttaatactctgagggcaggcttctgtgaaaaggttgcctgggctcagtgtgagattttgccataaaaaggggtcctgcccctgtgtacagacagatcggaatctagagtgcatactcagagtccccgcggttccggggctctgatctcagggcatctttgcctagagatcctctacgccggacgcatcgtggccgggtaccgagctcgaattcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgcc

SEQ ID NO: 27 - protein, 510 aa, Artificial Sequence
  CD19 CAR2-63a, mature polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAKPTTTPAPRPPTPAPTIASQPLSLRPEACRPAAGGAVHTRGLDFAPRKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR